(19) United States
(12) Patent Application Publication
(10) Pub. No.: US 2006/0134671 A1
(43) Pub. Date: Jun. 22, 2006

(54) Methods and systems for prognosis and treatment of solid tumors

(75) Inventors: Michael E. Burczynski, Collegeville, PA (US); Andrew J. Dorner, Lexington, MA (US); Natalie C. Twine, Goffstown, NH (US); William L. Trepicchio, Andover, MA (US); Donna K. Slonim, North Andover, MA (US); Andrew Strahs, Maynard, MA (US); Frederick Immermann, Suffern, NY (US)

Correspondence Address: KIRKPATRICK & LOCKHART NICHOLSON GRAHAM LLP/WYETH, STATE STREET FINANCIAL CENTER, ONE LINCOLN STREET, BOSTON, MA, 02111-2950, US

(73) Assignee: Wyeth, Madison, NJ (US)

(21) Appl. No.: 11/285,502

(22) Filed: Nov. 22, 2005

Related U.S. Application Data
(60) Provisional application No. 60/629,681, filed on Nov. 22, 2004.

(51) Int. Cl. C12Q 1/68; G06F 19/00

(52) U.S. Cl. 435/6; 702/20

(57) ABSTRACT

The present invention provides methods, systems and equipment for the prognosis and treatment of renal cell carcinoma (RCC) or other solid tumors. Genes prognostic of clinical outcomes of a solid tumor can be identified according to the present invention. The expression profiles of these genes in peripheral blood mononuclear cells (PBMCs) of patients who have the solid tumor are correlated with clinical outcome of these patients. Examples of RCC prognosis genes are illustrated in Tables 2 and 3. These genes can be used as surrogate markers for predicting clinical outcome of an RCC patient of interest. These genes can also be used for the selection of a favorable treatment for an RCC patient of interest.

### CROSS-REFERENCE TO RELATED APPLICATIONS

The present application claims priority to U.S. application 60/629,681, filed Nov. 22, 2004, and incorporates by reference its entire disclosure as well as the entire disclosures of U.S. application Ser. Nos. 10/834,268, filed Apr. 29, 2004; 60/538,246, filed Jan. 23, 2004; and 60/466,067, filed Apr. 29, 2003.

### TECHNICAL FIELD

The present invention relates to solid tumor prognosis genes and methods of using the same for the prognosis and treatment of solid tumors.

### BACKGROUND

Expression profiling studies in primary tissues have demonstrated that there exist transcriptional differences between normal and malignant tissues. See, for example, Su, et al., CANCER RES, 61:7388-7393 (2001); and Ramaswamy, et al., PROC NATL ACAD SCI U.S.A., 98:15149-15151 (2001). Recent clinical analyses have also identified expression profiles within tumors that appear to be highly correlated with certain measures of clinical outcomes. One study has demonstrated that expression profiling of primary tumor biopsies yields prognostic “signatures” that rival or may even out-perform currently accepted standard measures of risk in cancer patients. See van de Vijver, et al., N ENGL J MED, 347:1999-2009 (2002).

### SUMMARY OF THE INVENTION

The present invention provides methods, systems and equipment for prognosis and treatment of renal cell carcinoma (RCC) or other solid tumors. Genes prognostic of clinical outcomes of a solid tumor can be identified by the present invention. The expression profiles of these genes in peripheral blood mononuclear cells (PBMCs) of patients who have the solid tumor are correlated with clinical outcome of these patients. These genes can be used as surrogate markers for predicting clinical outcome of a patient of interest who has the solid tumor. These genes can also be used to identify or select treatments that can produce favorable outcomes for the patient of interest.

In one aspect, the present invention provides methods useful for the prognosis or selection of treatment of a solid tumor in a patient of interest. The methods include comparing an expression profile of one or more prognosis genes in a peripheral blood sample of the patient of interest to at least one reference expression profile of the prognosis gene(s), where each of the prognosis gene(s) is differentially expressed in PBMCs of a first class of patients as compared to PBMCs of a second class of patients. Both the first and second classes of patients have the same solid tumor as the patient of interest, but each class of patients has a different clinical outcome. In many embodiments, the prognosis gene(s) includes at least one gene whose pretreatment expression profile in PBMCs of the two classes of patients, as determined by Affymetrix HG-U133A genechips, is correlated with a class distinction under a class-based correlation analysis (e.g., the nearest-neighbor analysis or the significance method of microarrays method), where the class distinction represents an idealized expression pattern of the gene in PBMCs of the two classes of patients.

Solid tumors amenable to the present invention include, but are not limited to, RCC, prostate cancer, head/neck cancer, and other tumors that do not have their origins in blood or lymph cells. Clinical outcome can be measured by any clinical indicator. In one embodiment, clinical outcome is measured by time to disease progression (TTP) or time to death (TTD). Other patient responses to a therapeutic treatment, such as complete response, partial response, minor response, stable disease, progressive disease, non-progressive disease, or any combination thereof, can also be used to measure clinical outcome. Examples of solid tumor treatments amenable to the present invention include, but are not limited to, drug therapy (e.g., CCI-779 therapy), chemotherapy, hormone therapy, radiotherapy, immunotherapy, surgery, gene therapy, anti-angiogenesis therapy, palliative therapy, or any combination thereof.

The peripheral blood sample of the patient of interest can be a whole blood sample, or a blood sample comprising enriched or purified PBMCs. Other types of blood samples can also be used in the present invention. In many cases, the peripheral blood samples used to prepare the expression profile of the patient of interest and the reference expression profile(s) are baseline samples isolated prior to a therapeutic treatment of the patients.

The reference expression profile(s) can include an average expression profile of the prognosis gene(s) in peripheral blood samples of patients who have the same solid tumor as the patient of interest and whose clinical outcome is known or determinable. The reference expression profile(s) can also include a set of individual expression profiles each of which represents the peripheral blood expression pattern of the prognosis gene(s) in a particular reference patient who has the same solid tumor as the patient of interest and know or determinable clinical outcome. Other types of reference expression profiles can also be used in the present invention. In many cases, the expression profile of the patient of interest and the reference expression profile(s) are prepared using the same or comparable methodologies.

Any comparison method can be used to compare the expression profile of the patient of interest to the reference expression profile(s). In one embodiment, the comparison is based on the absolute or relative peripheral blood expression level of each prognosis gene. In another embodiment, the comparison is based on the ratios between expression levels of two or more prognosis genes. In yet another embodiment, the comparison is carried out by using methods such as the k-nearest-neighbors algorithm or the weighted-voting algorithm.

In one embodiment, the patient of interest being evaluated has RCC, and clinical outcome is measured by patient response to a CCI-779 therapy. Examples of RCC prognosis genes are depicted in Tables 2 and 3. In one example, the RCC prognosis gene(s) employed in the outcome prediction comprises at least one gene selected from Table 2. In many cases, the RCC prognosis genes comprise two or more genes selected from Table 2, such as at least one gene selected from Gene Nos. 1-7 and at least another gene selected from Gene Nos. 8-14. Gene or genes thus selected can be used to predict TTD of an RCC patient of interest. In another example, the RCC prognosis gene(s) employed in the outcome prediction comprises at least one gene selected from Table 3. In many instances, the RCC prognosis genes comprise two or more genes selected from Table 3, such as at least one gene selected from Gene Nos. 1-14 and at least another gene selected from Gene Nos. 15-28. Genes or genes thus selected can be used to predict TTP of the RCC patient of interest. In a further example, the RCC prognosis genes employed in the outcome prediction include a classifier selected from Table 4, and the expression profile of the RCC patient of interest is compared to the reference expression profiles using a k-nearest-neighbors algorithm or a weighted-voting algorithm.

The present invention also features systems useful for the prognosis or selection of treatment of a solid tumor in a patient of interest. The systems include (1) a first storage medium comprising data that represent an expression profile of one or more prognosis genes in a peripheral blood sample of a patient of interest, (2) a second storage medium comprising data that represent at least one reference expression profile of the prognosis gene(s), (3) a program capable of comparing the expression profile of the patient of interest to the reference expression profile, and (4) a processor capable of executing the program. The expression levels of the prognosis genes in PBMCs of patients having the solid tumor, as measured by Affymetrix HG-U133A genechips, are correlated with clinical outcomes of the patients. In one embodiment, the patient of interest has RCC, and the prognosis genes are selected from Tables 2 and 3.

In addition, the present invention features kits useful for the prognosis or selection of treatment of a solid tumor in a patient of interest. Each kit includes at least one probe for a solid tumor prognosis gene, such as an RCC prognosis gene selected from Tables 2 and 3.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The drawings are provided for illustration, not limitation.

FIG. 1A demonstrates the accuracy of nearest-neighbor classifiers with increasing size (from 2 to 200) for predicting long versus short TTD under leave-one-out cross validation. The smallest optimally-predictive model with the highest accuracy was a six-gene classifier, providing 71% overall accuracy, and is marked with an arrow in the Figure.

FIG. 1B shows the accuracy of nearest-neighbor classifiers with increasing size (from 2 to 200) for predicting long versus short TTD under 10-fold cross validation. The smallest optimally-predictive model with the highest accuracy was a 14-gene classifier, providing 71% overall accuracy, and is marked with an arrow in the Figure.

FIG. 1C illustrates the accuracy of nearest-neighbor classifiers with increasing size (from 2 to 200) for predicting long versus short TTD under 4-fold cross validation. The smallest optimally-predictive model with the highest accuracy was a 14-gene classifier, providing 69% overall accuracy, and is marked with an arrow in the Figure.

FIG. 2A depicts the accuracy of nearest-neighbor classifiers with increasing size for predicting long versus short TTP under leave-one-out cross validation. The smallest optimally-predictive model with the highest accuracy was an 8-gene classifier, providing 86% overall accuracy, and is marked with an arrow in the Figure.

FIG. 2B shows the accuracy of nearest-neighbor classifiers with increasing size for predicting long versus short TTP under 10-fold cross validation. The smallest optimally-predictive model with the highest accuracy was a 28-gene classifier, providing 88% overall accuracy, and is marked with an arrow in the Figure.

FIG. 2C illustrates the accuracy of nearest-neighbor classifiers with increasing size for predicting long versus short TTP under 4-fold cross validation. The smallest optimally-predictive model with the highest accuracy was an 8-gene classifier, providing 88% overall accuracy, and is marked with an arrow in the Figure.

### DETAILED DESCRIPTION

The present invention provides methods for prognosis and selection of treatment of RCC or other solid tumors. These methods employ prognosis genes that are differentially expressed in peripheral blood samples of solid tumor patients who have different clinical outcomes. The peripheral blood expression profiles of many of these prognosis genes are correlated with patients' clinical outcome under a class-based correlation model. In many embodiments, the solid tumor patients can be divided into at least two classes based on their clinical outcome, and the pretreatment PBMC expression profiles of the prognosis genes are correlated with a class distinction under a neighborhood analysis, where the class distinction represents an idealized expression pattern of these genes in PBMCs of the two classes of patients.

The prognosis genes of the present invention can be used as surrogate markers for the prediction of clinical outcome of patients having RCC or other solid tumors. The prognosis genes of the present invention can also be used for the identification or selection of favorable treatments of RCC or other solid tumors. Different patients may have distinct clinical responses to a therapeutic treatment due to individual heterogeneity of the molecular mechanism of the disease. The identification of gene expression patterns that correlate with patient response allows clinicians to select treatments based on predicted patient responses and thereby avoid adverse reactions. This provides improved power and safety of clinical trials and increased benefit/risk ratio for drugs and other therapeutic treatments. Peripheral blood is a tissue that can be routinely obtained from patients in a minimally invasive manner. By determining the correlation between patient outcome and gene expression profiles in peripheral blood samples, the present invention represents a significant advance in clinical pharmacogenomics and solid tumor treatment.

Various aspects of the invention are described in further detail in the following subsections. The use of subsections is not meant to limit the invention. Each subsection may apply to any aspect of the invention. In this application, the use of “or” means “and/or” unless stated otherwise.

### I. General Methods for Identifying Solid Tumor Prognosis Genes

Previous studies demonstrated that baseline expression profiles in PBMCs from solid tumor patients were significantly distinct from those of disease-free subjects. See U.S. patent application Ser. No. 10/717,597, filed Nov. 21, 2003, U.S. Provisional Application Ser. No. 60/459,782, filed Apr. 3, 2003, and U.S. Provisional Application Ser. No. 60/427,982, filed Nov. 21, 2002, all of which are incorporated herein by reference. Studies also showed that gene expression profiles in PBMCs were predictive of anti-cancer drug activity in vivo. See U.S. patent application Ser. No. 10/793,032, filed Mar. 5, 2004, U.S. patent application Ser. No. 10/775,169, filed Feb. 11, 2004, and U.S. Provisional Application Ser. No. 60/446,133, filed Feb. 11, 2003, all of which are also incorporated herein by reference. In addition, studies indicated that PBMC baseline expression profiles were correlated with clinical outcomes of RCC or other non-blood diseases. See U.S. patent application Ser. No. 10/834,114, filed Apr. 29, 2004, U.S. Provisional Patent Application Ser. No. 60/538,246, filed Jan. 23, 2004, and U.S. Provisional Patent Application Ser. No. 60/466,067, filed Apr. 29, 2003.

The present invention further evaluates the correlation between peripheral blood gene expression and clinical outcome of RCC or other solid tumors. Prognosis genes for RCC or other solid tumors can be identified according to the present invention. These genes are differentially expressed in peripheral blood samples of solid tumor patients who have different clinical outcomes. The peripheral blood expression profiles of many of these genes are correlated with a class distinction between patients of different outcome classes. In many embodiments, the peripheral blood expression profiles are baseline profiles representing peripheral blood gene expression prior to the initiation of treatment of the patients. The peripheral blood expression profiles can also be selected to represent gene expression during the course of the treatment. Correlation analyses suitable for the present invention include, but are not limited to, the nearest-neighbor analysis (Golub, et al., SCIENCE, 286: 531-537 (1999)), the significance method of microarrays (SAM) method (Tusher, et al., PROC. NATL. ACAD. SCI. U.S.A., 98:5116-5121 (2001)), or other class-based correlation metrics.

Solid tumors amenable to the present invention include, without limitation, RCC, prostate cancer, head/neck cancer, ovarian cancer, testicular cancer, brain tumor, breast cancer, lung cancer, colon cancer, pancreas cancer, stomach cancer, bladder cancer, skin cancer, cervical cancer, uterine cancer, and liver cancer. A solid tumor can be measured or evaluated using direct or indirect visualization procedures. Suitable visualization methods include, but are not limited to, scans (such as X-rays, computerized axial tomography (CT), magnetic resonance imaging (MRI), positron emission tomography (PET), or ultrasonography (U/S)), biopsy, palpation, endoscopy, laparoscopy, and other suitable means as appreciated by those skilled in the art.

Clinical outcome of a solid tumor can be assessed by numerous criteria. In many embodiments, clinical outcome is assessed based on patients' response to a therapeutic treatment. Examples of clinical outcome measures include, without limitation, complete response, partial response, minor response, stable disease, progressive disease, time to disease progression (TTP), time to death (TTD or Survival), or any combination thereof. Examples of solid tumor treatments include, without limitation, drug therapy (e.g., CCI-779 therapy), chemotherapy, hormone therapy, radiotherapy, immunotherapy, surgery, gene therapy, anti-angiogenesis therapy, palliative therapy, or other conventional or non-conventional therapies, or any combination thereof.

In one embodiment, clinical outcome is evaluated based on the WHO Reporting Criteria, such as those described in WHO Publication, No. 48 (World Health Organization, Geneva, Switzerland, 1979). Under the Criteria, uni- or bidimensionally measurable lesions are measured at each assessment. When multiple lesions are present in any organ, up to 6 representative lesions can be selected, if available.

In one example, clinical outcome is determined based on a classification system composed of clinical categories such as complete response, partial response, minor response, stable disease, progressive disease, or any combination thereof. “Complete response” (CR) means complete disappearance of all measurable and evaluable disease, determined by two observations not less than 4 weeks apart. There is no new lesion and no disease related symptom. “Partial response” (PR) in reference to bidimensionally measurable disease means decrease by at least about 50% of the sum of the products of the largest perpendicular diameters of all measurable lesions as determined by 2 observations not less than 4 weeks apart. “Partial response” in reference to unidimensionally measurable disease means decrease by at least about 50% in the sum of the largest diameters of all lesions as determined by 2 observations not less than 4 weeks apart. It is not necessary for all lesions to have regressed to qualify for partial response, but no lesion should have progressed and no new lesion should appear. The assessment should be objective. “Minor response” in reference to bidimensionally measurable disease means about 25% or greater decrease but less than about 50% decrease in the sum of the products of the largest perpendicular diameters of all measurable lesions. “Minor response” in reference to unidimensionally measurable disease means decrease by at least about 25% but less than about 50% in the sum of the largest diameters of all lesions.

“Stable disease” (SD) in reference to bidimensionally measurable disease means less than about 25% decrease or less than about 25% increase in the sum of the products of the largest perpendicular diameters of all measurable lesions. “Stable disease” in reference to unidimensionally measurable disease means less than about 25% decrease or less than about 25% increase in the sum of the diameters of all lesions. No new lesions should appear. “Progressive disease” (PD) refers to a greater than or equal to about a 25% increase in the size of at least one bidimensionally (product of the largest perpendicular diameters) or unidimensionally measurable lesion or appearance of a new lesion. The occurrence of pleural effusion or ascites is also considered as progressive disease if this is substantiated by positive cytology. Pathological fracture or collapse of bone is not necessarily evidence of disease progression.

In yet another embodiment, overall subject tumor response for uni- and bidimensionally measurable disease is determined according to Table 1.

TABLE 1
Overall Subject Tumor Response

| Response in | Response in | |
|---|---|---|
| Bidimensionally | Unidimensionally | Overall Subject |
| Measurable Disease | Measurable Disease | Tumor Response |
| PD | Any | PD |
| Any | PD | PD |
| SD | SD or PR | SD |
| SD | CR | PR |
| PR | SD or PR or CR | PR |
| CR | SD or PR | PR |
| CR | CR | CR |

Overall subject tumor response for non-measurable disease can be assessed, for instance, in the following situations:

a) Overall complete response: if non-measurable disease is present, it should disappear completely. Otherwise, the subject cannot be considered as an “overall complete responder.”

b) Overall progression: in case of a significant increase in the size of non-measurable disease or the appearance of a new lesion, the overall response will be progression.

Clinical outcome can also be assessed by other criteria. For instance, clinical outcome can be measured by TTP or TTD. TTP refers to the interval from the date of initiation of a therapeutic treatment until the first day of measurement of progressive disease. TTD refers to the interval from the date of initiation of a therapeutic treatment to the time of death, or censored at the last date known alive.

Solid tumor patients can be classified based on their respective clinical outcomes. Solid tumor patients can also be classified by using traditional clinical risk assessment methods. In many cases, these risk assessment methods employ a number of prognostic factors to classify patients into different prognosis or risk groups. One example is Motzer risk assessment for RCC, as described in Motzer, et al., J CLIN ONCOL, 17:2530-2540 (1999). Patients in different risk groups may have different responses to a therapy.

In many cases, the peripheral blood samples used for the identification of the prognosis genes are “baseline” or “pretreatment” samples. These samples are isolated from respective patients prior to a therapeutic treatment and can be used to identify genes whose baseline peripheral blood expression profiles are correlated with patient outcome in response to the treatment. Peripheral blood samples isolated at other treatment or disease stages can also be used to identify solid tumor prognosis genes.

A variety of types of peripheral blood samples can be used in the present invention. In one embodiment, the peripheral blood samples are whole blood samples. In another embodiment, the peripheral blood samples comprise enriched PBMCs. By “enriched,” it means that the percentage of PBMCs in the sample is higher than that in whole blood. In some cases, the PBMC percentage in an enriched sample is at least 1, 2, 3, 4, 5 or more times higher than that in whole blood. In some other cases, the PBMC percentage in an enriched sample is at least 90%, 95%, 98%, 99%, 99.5%, or more. Blood samples containing enriched PBMCs can be prepared using any method known in the art, such as Ficoll gradients centrifugation or CPTs (cell purification tubes).

The relationship between peripheral blood gene expression profiles and patient outcome can be evaluated by using global gene expression analyses. Methods suitable for this purpose include, but are not limited to, nucleic acid arrays (such as cDNA or oligonucleotide arrays), 2-dimensional SDS-polyacrylamide gel electrophoresis/mass spectrometry, and other high throughput nucleotide or polypeptide detection techniques.

Nucleic acid arrays allow for quantitative detection of the expression levels of a large number of genes at one time. Examples of nucleic acid arrays include, but are not limited to, Genechip® microarrays from Affymetrix (Santa Clara, Calif.), cDNA microarrays from Agilent Technologies (Palo Alto, Calif.), and bead arrays described in U.S. Pat. Nos. 6,288,220 and 6,391,562.

The polynucleotides to be hybridized to a nucleic acid array can be labeled with one or more labeling moieties to allow for detection of hybridized polynucleotide complexes. The labeling moieties can include compositions that are detectable by spectroscopic, photochemical, biochemical, bioelectronic, immunochemical, electrical, optical or chemical means. Exemplary labeling moieties include radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like. Unlabeled polynucleotides can also be employed. The polynucleotides can be DNA, RNA, or a modified form thereof.

Hybridization reactions can be performed in absolute or differential hybridization formats. In the absolute hybridization format, polynucleotides derived from one sample, such as PBMCs from a patient in a selected outcome class, are hybridized to the probes on a nucleic acid array. Signals detected after the formation of hybridization complexes correlate to the polynucleotide levels in the sample. In the differential hybridization format, polynucleotides derived from two biological samples, such as one from a patient in a first outcome class and the other from a patient in a second outcome class, are labeled with different labeling moieties. A mixture of these differently labeled polynucleotides is added to a nucleic acid array. The nucleic acid array is then examined under conditions in which the emissions from the two different labels are individually detectable. In one embodiment, the fluorophores Cy3 and Cy5 (Amersham Pharmacia Biotech, Piscataway N.J.) are used as the labeling moieties for the differential hybridization format.

Signals gathered from a nucleic acid array can be analyzed using commercially available software, such as those provided by Affymetrix or Agilent Technologies. Controls, such as for scan sensitivity, probe labeling and cDNA/cRNA quantitation, can be included in the hybridization experiments. In many embodiments, the nucleic acid array expression signals are scaled or normalized before being subject to further analysis. For instance, the expression signals for each gene can be normalized to take into account variations in hybridization intensities when more than one array is used under similar test conditions. Signals for individual polynucleotide complex hybridization can also be normalized using the intensities derived from internal normalization controls contained on each array. In addition, genes with relatively consistent expression levels across the samples can be used to normalize the expression levels of other genes. In one embodiment, the expression levels of the genes are normalized across the samples such that the mean is zero and the standard deviation is one. In another embodiment, the expression data detected by nucleic acid arrays are subject to a variation filter which excludes genes showing minimal or insignificant variation across all samples.

The gene expression data collected from nucleic acid arrays can be correlated with clinical outcome using a variety of methods. Suitable correlation methods include, but are not limited to, statistical methods (such as Spearman's rank correlation, Cox proportional hazard regression model, ANOVA/t test, or other suitable rank tests or survival models) and class-based correlation metrics (such as nearest-neighbor analysis).

In one embodiment, patients with a specified solid tumor are divided into at least two classes based on their clinical stratifications. The correlation between peripheral blood gene expression (e.g., PBMC gene expression profiles) and clinical outcome is analyzed by a supervised cluster or learning algorithm. Exemplary supervised clustering or learning algorithms include, but are not limited to, nearest-neighbor analysis, support vector machines, the SAM method, artificial neural networks, and SPLASH. Under the supervised algorithms, clinical outcome of each class of patients is either known or determinable. Genes that are differentially expressed in peripheral blood cells (e.g., PBMCs) of one class of patients compared to another class of patients can be identified. In many cases, the genes thus identified are substantially correlated with a class distinction between the two classes of patients. The genes thus identified can be used as surrogate markers for predicting clinical outcome of the solid tumor in a patient of interest.

In another embodiment, patients with a specified solid tumor are divided into at least two classes based on their peripheral blood gene expression profiles. Methods suitable for this purpose include unsupervised clustering algorithms, such as self-organized maps (SOMs), k-means, principal component analysis, and hierarchical clustering. A substantial number (e.g., at least 50%, 60%, 70%, 80%, 90%, or more) of patients in one class may have a first clinical outcome, and a substantial number of patients in another class may have a second clinical outcome. Genes that are differentially expressed in the peripheral blood cells of one class of patients relative to another class of patients can be identified. These genes are also prognosis genes for the solid tumor.

In one example, patients with a specified solid tumor can be divided into three or more classes based on their clinical stratifications or peripheral blood gene expression profiles. Multi-class correlation metrics can be employed to identify genes that are differentially expressed in these classes. Exemplary multi-class correlation metrics include, but are not limited to, those employed by GeneCluster 2 software provided by MIT Center for Genome Research at Whitehead Institute (Cambridge, Mass.).

In a further embodiment, nearest-neighbor analysis (also known as neighborhood analysis) is used to analyze gene expression data gathered from nucleic acid arrays. The algorithm for neighborhood analysis is described in Golub, et al., SCIENCE, 286: 531-537 (1999), Slonim, et al., PROCS. OF THE FOURTH ANNUAL INTERNATIONAL CONFERENCE ON COMPUTATIONAL MOLECULAR BIOLOGY, Tokyo, Japan, April 8-11, p263-272 (2000), and U.S. Pat. No. 6,647,341, all of which are incorporated herein by reference. According to one version of the neighborhood analysis, the expression profile of each gene can be represented by an expression vector g=(e₁, e₂, e₃, . . . , eₙ), where eᵢ corresponds to the expression level of gene “g” in the ith sample. A class distinction can be represented by an idealized expression pattern c=(c₁, c₂, c₃, . . . , cₙ), where cᵢ=1 or −1, depending on whether the ith sample is isolated from class 0 or class 1. Class 0 may include patients having a first clinical outcome, and class 1 includes patients having a second clinical outcome. Other forms of class distinction can also be employed. Typically, a class distinction represents an idealized expression pattern, where the expression level of a gene is uniformly high for samples in one class and uniformly low for samples in the other class.

The correlation between gene “g” and the class distinction can be measured by a signal-to-noise score:
P(g,c)=[μ₁(g)−μ₂(g)]/[σ₁(g)+σ₂(g)]
where μ₁(g) and μ₂(g) represent the means of the log-transformed expression levels of gene “g” in class 0 and class 1, respectively, and σ₁(g) and σ₂(g) represent the standard deviation of the log-transformed expression levels of gene “g” in class 0 and class 1, respectively. A higher absolute value of a signal-to-noise score indicates that the gene is more highly expressed in one class than in the other. In one example, the samples used to derive the signal-to-noise scores comprise enriched or purified PBMCs and, therefore, the signal-to-noise score P(g,c) represents a correlation between the class distinction and the expression level of gene “g” in PBMCS.

The correlation between gene “g” and the class distinction can also be measured by other methods, such as by the Pearson correlation coefficient or the Euclidean distance, as appreciated by those skilled in the art.

The significance of the correlation between peripheral blood gene expression profiles and the class distinction can be evaluated using a random permutation test. An unusually high density of genes within the neighborhoods of the class distinction, as compared to random patterns, suggests that many genes have expression patterns that are significantly correlated with the class distinction. The correlation between genes and the class distinction can be diagrammatically viewed through a neighborhood analysis plot, in which the y-axis represents the number of genes within various neighborhoods around the class distinction and the x-axis indicates the size of the neighborhood (i.e., P(g,c)). Curves showing different significance levels for the number of genes within corresponding neighborhoods of randomly permuted class distinctions can also be included in the plot.

In many embodiments, the prognosis genes employed in the present invention are substantially correlated with a class distinction between two outcome classes. For instance, the prognosis genes employed in the present invention can be above the median significance level in the neighborhood analysis plot. This means that the correlation measure P(g,c) for each prognosis gene is such that the number of genes within the neighborhood of the class distinction having the size of P(g,c) is greater than the number of genes within the corresponding neighborhoods of randomly permuted class distinctions at the median significance level. For another instance, the prognosis genes employed in the present invention can be above the 10%, 5%, 2%, or 1% significance level. As used herein, x % significance level means that x % of random neighborhoods contain as many genes as the real neighborhood around the class distinction.

Class predictors can be constructed using the prognosis genes of the present invention. These class predictors are useful for assigning a class membership to a solid tumor patient of interest. In one embodiment, the prognosis genes in a class predictor are limited to those shown to be significantly correlated with the class distinction by the permutation test, such as those at above the 1%, 2%, 5%, 10%, 20%, 30%, 40%, or 50% significance level. In another embodiment, the expression level of each prognosis gene in a class predictor is substantially higher or substantially lower in PBMCs of one class of patients than in the other class of patients. In still another embodiment, the prognosis genes in a class predictor have top absolute values of P(g,c). In yet another embodiment, the p-value under a Student's t-test (e.g., two-tailed distribution, two sample unequal variance) for each prognosis gene in a class predictor is no more than 0.05, 0.01, 0.005, 0.001, 0.0005, 0.0001, or less. The p-value suggests the statistical significance of the difference between the PBMC expression profile of a prognosis gene in one class of patients and that in another class of patients. Lesser p-values indicate more statistical significance for the differences observed between different classes of solid tumor RCC patients.

The SAM method can also be used to correlate peripheral blood gene expression profiles with clinical outcome classes. The prediction analysis of microarrays (PAM) method can then be used to identify gene sets that can best characterize a predefined outcome class and predict the class membership of new samples. See Tibshirani, et al., PROC. NATL. ACAD. SCI. U.S.A., 99:6567-6572 (2002).

In many embodiments, a class predictor of the present invention has at least 50% prediction accuracy under leave-one-out cross validation, 10-fold cross validation, or 4-fold cross validation. In a typical k-fold cross validation, the data is divided into k subsets of approximately equal size. The model is trained k times, each time leaving out one of the subsets from training and using the omitted subset as the test samples to calculate the prediction error. If k equals the sample size, it becomes the leave-one-out cross validation. In many instances, a class predictor of the present invention has at least 60%, 70%, 80%, 90%, 95%, or 99% accuracy under leave-one-out cross validation, 10-fold cross validation, or 4-fold cross validation.

Other class-based correlation metrics or statistical methods can also be used to identify prognosis genes whose expression profiles in peripheral blood samples are correlated with clinical outcome of solid tumor patients. Many of these methods can be performed by using public or commercial softwares.

Other methods capable of identifying solid tumor prognosis genes include, but are not limited, RT-PCR, Northern Blot, in situ hybridization, and immunoassays such as ELISA, RIA or Western Blot. These genes are differentially expressed in peripheral blood cells (e.g., PBMCs) of one class of patients relative to another class of patients. In many cases, the average peripheral blood expression level of each of these genes in one class of patients is statistically different from that in another class of patients. For instance, the p-value under an appropriate statistical significance test (e.g., Student's t-test) for the observed difference can be no more than 0.05, 0.01, 0.005, 0.001, 0.0005, 0.0001, or less. In many other cases, each prognosis gene thus identified has at least 2-, 3-, 4-, 5-, 10-, or 20-fold difference in the average PBMC expression level between one class of patients and another class of patients.

Prognosis genes for other non-blood diseases can be similarly identified according to the present invention, where the correlation between the peripheral blood expression profiles of these genes and patient outcome is statistically significant. The peripheral blood expression patterns of these prognosis genes are therefore indicative of clinical outcome of patients having these non-blood diseases.

### II. Identification of RCC Prognosis Genes Using HG-U133A Microarrays

RCC comprises the majority of all cases of kidney cancer and is one of the ten most common cancers in industrialized countries, comprising 2% of adult malignancies and 2% of cancer-related deaths. Several prognostic factors and scoring indices have been developed for patients diagnosed with RCC, typified by multivariate assessments of several key indicators. As an example, one prognostic scoring system employs the five prognostic factors proposed by Motzer, et al., J CLIN ONCOL, 17:2530-2540 (1999)—namely, Karnofsky performance status, serum lactate dehydrognease, hemoglobin, serum calcium, and presence/absence of prior nephrectomy.

The present invention identifies RCC prognosis genes whose peripheral blood expression profiles correlate with patient outcome in CCI-779 therapy. The cytostatic mTOR inhibitor CCI-779 was evaluated in RCC patients for its anti-cancer effect. PBMCs collected prior to CCI-779 therapy were analyzed on oligonucleotide arrays (HG-U133A, Affymetrix, Santa Clara, Calif., USA) in order to determine whether mononuclear cells from RCC patients possessed transcriptional patterns predictive of patient outcome.

PBMCs were isolated prior to CCI-779 therapy from peripheral blood of 45 advanced RCC patients (18 females and 27 males) participating in a phase 2 clinical trial study. Written informed consent for the pharmacogenomic portion of the clinical study was received for all individuals and the project was approved by the local Institutional Review Boards at the participating clinical sites. RCC tumors of patients were classified at the clinical sites as conventional (clear cell) carcinomas (24), granular (1), papillary (3), or mixed subtypes (7). Ten tumors were classified as unknown. RCC patients were primarily of Caucasian descent (44 Caucasian, 1 African-American) and had a mean age of 58 years (range of 40-78 years). Inclusion criteria included patients with histologically confirmed advanced renal cancer who had received prior therapy for advanced disease, or who had not received prior therapy for advanced disease but were not appropriate candidates to receive high doses of IL-2 therapy. Other inclusion criteria included patients with (1) bi-dimensionally measurable evidence of disease; (2) evidence of progression of the disease prior to study entry; (3) an age of 18 years or older; (4) ANC >1500 μL, platelet >100,000 μL and hemoglobin >8.5 g/dL; (5) adequate renal function evidenced by serum creatinine <1.5×upper limit of normal; (6) adequate hepatic function evidenced by biliruubin <1.5×upper limit of normal and AST <3×upper limit of normal (or AST <5×upper limit of normal if liver metastases were present); (7) serum cholesterol <350 mg/dL, triglycerides <300 mg/dL; (8) ECOG performance status 0-1; and (9) a life expectancy of at least 12 weeks. Exclusion criteria included patients who had (1) the presence of known CNS metastases; (2) surgery or radiotherapy within 3 weeks of start of dosing; (3) chemotherapy or biologic therapy for RCC within 4 weeks of start of dosing; (4) treatment with a prior investigational agent within 4 weeks of start of dosing; (5) immunocompromised status including those known to be HIV positive, or receiving concurrent use of immunosuppressive agents including corticosteroids; (6) active infections; (7) required treatment with anticonvulsant therapy; (8) presence of unstable angina/myocardial infarction within 6 months/ongoing treatment of life-threatening arrythmia; (9) history of prior malignancy in past 3 years; (10) hypersensitivity to macrolide antibiotics; and (11) pregnancy or any other illness which would substantially increase the risk associated with participation in the study.

These advanced RCC patients were treated with one of 3 doses of CCI-779 (25 mg, 75 mg, or 250 mg) administered as a 30 minute intravenous (IV) infusion once weekly for the duration of the trial. CCI-779 is an ester analog of the immunosuppressant rapamycin and as such is a potent, selective inhibitor of the mammalian target of rapamycin. The mammalian target of rapamycin (mTOR) activates multiple signaling pathways, including phosphorylation of p70s6kinase, which results in increased translation of 5′ TOP mRNAs encoding proteins involved in translation and entry into the G1 phase of the cell cycle. By virtue of its inhibitory effects on mTOR and cell cycle control, CCI-779 functions as a cytostatic and immunosuppressive agent.

Clinical staging and size of residual, recurrent or metastatic disease were recorded prior to treatment and every 8 weeks following initiation of CCI-779 therapy. Tumor size was measured in centimeters and reported as the product of the longest diameter and its perpendicular. Measurable disease was defined as any bidimensionally measurable lesion where both diameters >1.0 cm by CT-scan, X-ray or palpation. Tumor response was determined by the sum of the products of all measurable lesions. The categories for assignment of clinical response were given by the clinical protocol definitions (i.e., progressive disease, stable disease, minor response, partial response, and complete response). The category for assignment of prognosis under the Motzer risk assessment (favorable vs intermediate vs poor) was also used. Among the 45 RCC patients, 6 were assigned a favorable risk assessment, 17 patients possessed an intermediate risk score, and 22 patients received a poor prognosis classification. In addition to the categorical classifications, overall survival and time to disease progression were also monitored as clinical endpoints.

Baseline samples were isolated from the 45 RCC patients prior to the CCI-779 therapy. Of the 45 baseline samples, 42 were hybridized to HG-U133A genechips according to the manufacturer's guidelines. See GeneChip® Expression Analysis—Technical Manual (Part No. 701021 Rev. 3, Affymetrix, Inc. 1999-2002), the entire content of which is incorporated herein by reference. Signals were calculated from probe intensities by the MAS 5 algorithm, and signal intensities were converted to frequencies using the scale frequency normalization method as described in the Examples.

All PBMC profiles hybridized to U133A genechips were divided into categories based on clinical stratifications of 106-day TTP or year-long TTD. Several cross validation procedures, including leave one out cross validation, ten-fold cross validation and four-fold cross validation, were employed in order to assess the accuracy of class assignment. A nearest neighbors algorithm was used to generate gene classifiers of increasing size, which were evaluated by the various cross validation approaches. The classifiers that gave the highest accuracy of class assignment by each of the 3 cross validation approaches were identified.

Specifically, classifiers consisting of genes selected from Tables 2 or 3 were built and evaluated for prediction accuracy by the 3 cross validation methods. Examples of these classifiers are illustrated in Table 4. Classifiers 1-7 were evaluated for discrimination of patients with short (less than 365 days) versus longer (greater than 365 days) survival, and classifiers 8-21 were assessed for discrimination of patients with short (less than 106 days) versus longer (greater than 106 days) TTP.

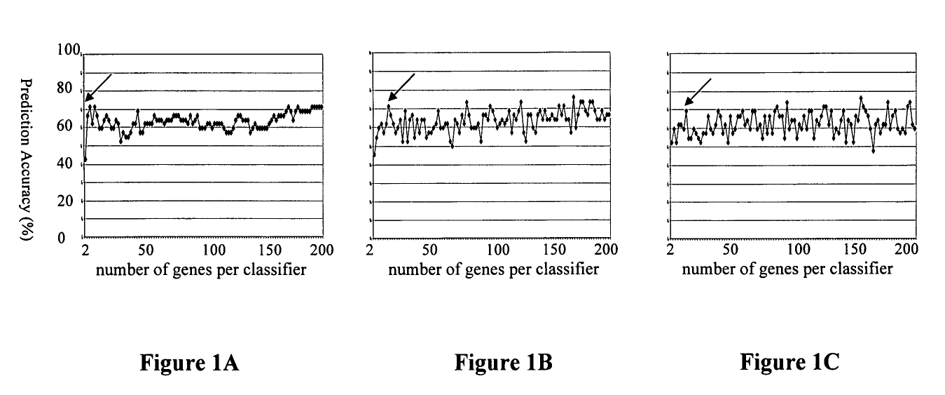

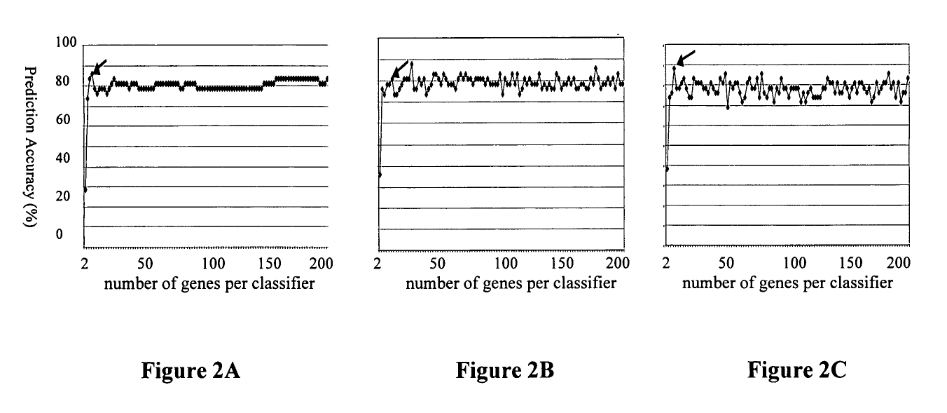

For both clinical stratifications, the three cross validation approaches gave similar accuracies of class assignment. The results of the cross validation analyses of the 42 PBMC samples hybridized to U133A chips for classification on the basis of year-long survival are depicted in FIGS. 1A-1C, and for classification on the basis of 3-month TTP in FIGS. 2A-2C. A few transcript sequences in the gene classifiers based on the training set of samples hybridized to the Affymetrix HG-U95A chips (see, e.g., Tables 20 and 21 in U.S. patent application Ser. No. 10/834,114) were also present in Tables 2 and 3 (e.g., protein kinase C delta as highly correlated with short-term TTP, and MD-2 protein as highly correlated with shorter survival).

TABLE 2
Genes for Discrimination of Patients Experiencing Short (Less Than
One Year) vs Longer (Greater Than One Year) Survival

| Gene No. | Patient Class | Qualifier | Gene Symbol | Gene Description | Unigene |
|---|---|---|---|---|---|
| 1 | Less_than_365_TTD | 217972_at | FLJ20420 | hypothetical protein FLJ20420 | Hs.6693 |
| 2 | Less_than_365_TTD | 201989_s_at | CREBL2 | cAMP responsive element | Hs.13313 |
| | | | | binding protein-like 2 | |
| 3 | Less_than_365_TTD | 209482_at | RPP20 | POP7 (processing of | Hs.18747 |
| | | | | precursor, S. cerevisiae) | |
| | | | | homolog | |
| 4 | Less_than_365_TTD | 210186_s_at | FKBP1A | FK506 binding protein 1A | Hs.179661 |
| | | | | (12 kD) | |
| 5 | Less_than_365_TTD | 206584_at | MD-2 | MD-2 protein | Hs.69328 |
| 6 | Less_than_365_TTD | 200785_s_at | LRP1 | low density lipoprotein-related | Hs.89137 |
| | | | | protein 1 (alpha-2- | |
| | | | | macroglobulin receptor) | |
| 7 | Less_than_365_TTD | 203645_s_at | CD163 | CD163 antigen | Hs.74076 |
| 8 | Greater_than_365_TTD | 215275_at | UNK_AW963138 | | |
| 9 | Greater_than_365_TTD | 202547_s_at | ARHGEF7 | Rho guanine nucleotide | Hs.172813 |
| | | | | exchange factor (GEF) 7 | |
| 10 | Greater_than_365_TTD | 221736_at | UNK_AA156777 | | Hs.25431 |
| 11 | Greater_than_365_TTD | 212231_at | FBXO21 | F-box only protein 21 | Hs.184227 |
| 12 | Greater_than_365_TTD | 211256_x_at | BTN2A1 | butyrophilin, subfamily 2, | Hs.169963 |
| | | | | member A1 | |
| 13 | Greater_than_365_TTD | 208723_at | USP11 | ubiquitin specific protease 11 | Hs.171501 |
| 14 | Greater_than_365_TTD | 208774_at | CSNK1D | casein kinase 1, delta | Hs.75852 |

TABLE 3
Genes for Discrimination of Patients Undergoing Short (Less
Than 106 Days) vs Longer (Greater Than 106 Days) TTP

| Gene No. | Patient Class | Qualifier | Gene Symbol | Gene Description | Unigene |
|---|---|---|---|---|---|
| 1 | Less_Than_106_TTP | 208918_s_at | FLJ13052 | NAD kinase | Hs.220324 |
| 2 | Less_Than_106_TTP | 214084_x_at | NCF1 | neutrophil cytosolic factor 1 (47 kD, chronic | Hs.1583 |
| | | | | granulomatous disease, autosomal 1) | |
| 3 | Less_Than_106_TTP | 202146_at | IFRD1 | interferon-related developmental regulator 1 | Hs.7879 |
| 4 | Less_Than_106_TTP | 202545_at | PRKCD | protein kinase C, delta | Hs.155342 |
| 5 | Less_Than_106_TTP | 211133_x_at | LILRB3 | leukocyte immunoglobulin-like receptor, | Hs.105928 |
| | | | | subfamily B (with TM and ITIM domains), | |
| | | | | member 3 | |
| 6 | Less_Than_106_TTP | 204961_s_at | NCF1 | neutrophil cytosolic factor 1 (47 kD, chronic | Hs.1583 |
| | | | | granulomatous disease, autosomal 1) | |
| 7 | Less_Than_106_TTP | 205483_s_at | ISG15 | interferon-stimulated protein, 15 kDa | Hs.432233 |
| 8 | Less_Than_106_TTP | 202086_at | MX1 | myxovirus (influenza virus) resistance 1, | Hs.76391 |
| | | | | interferon-inducible protein p78 (mouse) | |
| 9 | Less_Than_106_TTP | 203104_at | CSF1R | colony stimulating factor 1 receptor, formerly | Hs.174142 |
| | | | | McDonough feline sarcoma viral (v-fms) | |
| | | | | oncogene homolog | |
| 10 | Less_Than_106_TTP | 205922_at | VNN2 | vanin 2 | Hs.121102 |
| 11 | Less_Than_106_TTP | 213931_at | ID2 | inhibitor of DNA binding 2, dominant negative | Hs.180919 |
| | | | | helix-loop-helix protein | |
| 12 | Less_Than_106_TTP | 203514_at | MAP3K3 | mitogen-activated protein kinase kinase kinase 3 | Hs.29282 |
| 13 | Less_Than_106_TTP | 204336_s_at | RGS19 | regulator of G-protein signalling 19 | Hs.422336 |
| 14 | Less_Than_106_TTP | 221541_at | DKFZP434B044 | hypothetical protein DKFZp434B044 | Hs.262958 |
| 15 | Greater_Than_106_TTP | 217802_s_at | NUCKS | similar to rat nuclear ubiquitous casein kinase 2 | Hs.118064 |
| 16 | Greater_Than_106_TTP | 201019_s_at | EIF1A | eukaryotic translation initiation factor 1A | Hs.4310 |
| 17 | Greater_Than_106_TTP | 212231_at | FBXO21 | F-box only protein 21 | Hs.184227 |
| 18 | Greater_Than_106_TTP | 203156_at | AKAP11 | A kinase (PRKA) anchor protein 11 | Hs.232076 |
| 19 | Greater_Than_106_TTP | 206545_at | CD28 | CD28 antigen (Tp44) | Hs.1987 |
| 20 | Greater_Than_106_TTP | 218014_at | FLJ12549 | frount | Hs.184352 |
| 21 | Greater_Than_106_TTP | 203712_at | KIAA0020 | KIAA0020 gene product | Hs.2471 |
| 22 | Greater_Than_106_TTP | 221452_s_at | MGC1223 | hypothetical protein MGC1223 | Hs.273077 |
| 23 | Greater_Than_106_TTP | 212168_at | RBM12 | RNA binding motif protein 12 | Hs.180895 |
| 24 | Greater_Than_106_TTP | 213111_at | KIAA0981 | KIAA0981 protein | Hs.158135 |
| 25 | Greater_Than_106_TTP | 214669_x_at | IGKC | immunoglobulin kappa constant | Hs.406565 |
| 26 | Greater_Than_106_TTP | 219423_x_at | TNFRSF12 | tumor necrosis factor receptor superfamily, | Hs.180338 |
| | | | | member 12 (translocating chain-association | |
| | | | | membrane protein) | |
| 27 | Greater_Than_106_TTP | 204642_at | EDG1 | endothelial differentiation, sphingolipid G- | Hs.154210 |
| | | | | protein-coupled receptor, 1 | |
| 28 | Greater_Than_106_TTP | 201477_s_at | RRM1 | ribonucleotide reductase M1 polypeptide | Hs.2934 |

TABLE 4
Examples of Classifiers

| Classifier | Genes | Patient Class Predicted |
|---|---|---|
| 1 | Gene Nos. 1 and 8 of Table 2 | TTD of less than one year vs. TTD of greater than one year |
| 2 | Gene Nos. 1-2 and 8-9 of Table 2 | TTD of less than one year vs. TTD of greater than one year |
| 3 | Gene Nos. 1-3 and 8-10 of Table 2 | TTD of less than one year vs. TTD of greater than one year |
| 4 | Gene Nos. 1-4 and 8-11 of Table 2 | TTD of less than one year vs. TTD of greater than one year |
| 5 | Gene Nos. 1-5 and 8-12 of Table 2 | TTD of less than one year vs. TTD of greater than one year |
| 6 | Gene Nos. 1-6 and 8-13 of Table 2 | TTD of less than one year vs. TTD of greater than one year |
| 7 | Gene Nos. 1-14 of Table 2 | TTD of less than one year vs. TTD of greater than one year |
| 8 | Gene Nos. 1 and 15 of Table 3 | TTP of less than 106 days vs. TTP of greater than 106 days |
| 9 | Gene Nos. 1-2 and 15-16 of Table 3 | TTP of less than 106 days vs. TTP of greater than 106 days |
| 10 | Gene Nos. 1-3 and 15-17 of Table 3 | TTP of less than 106 days vs. TTP of greater than 106 days |
| 11 | Gene Nos. 1-4 and 15-18 of Table 3 | TTP of less than 106 days vs. TTP of greater than 106 days |
| 12 | Gene Nos. 1-5 and 15-19 of Table 3 | TTP of less than 106 days vs. TTP of greater than 106 days |
| 13 | Gene Nos. 1-6 and 15-20 of Table 3 | TTP of less than 106 days vs. TTP of greater than 106 days |
| 14 | Gene Nos. 1-7 and 15-21 of Table 3 | TTP of less than 106 days vs. TTP of greater than 106 days |
| 15 | Gene Nos. 1-8 and 15-22 of Table 3 | TTP of less than 106 days vs. TTP of greater than 106 days |
| 16 | Gene Nos. 1-9 and 15-23 of Table 3 | TTP of less than 106 days vs. TTP of greater than 106 days |
| 17 | Gene Nos. 1-10 and 15-24 of Table 3 | TTP of less than 106 days vs. TTP of greater than 106 days |
| 18 | Gene Nos. 1-11 and 15-25 of Table 3 | TTP of less than 106 days vs. TTP of greater than 106 days |
| 19 | Gene Nos. 1-12 and 15-26 of Table 3 | TTP of less than 106 days vs. TTP of greater than 106 days |
| 20 | Gene Nos. 1-13 and 15-27 of Table 3 | TTP of less than 106 days vs. TTP of greater than 106 days |
| 21 | Gene Nos. 1-28 of Table 3 | TTP of less than 106 days vs. TTP of greater than 106 days |

The pretreatment PBMC expression levels of the genes in Tables 2 and 3 are correlated with, and therefore predictive of, survival or time to progression in patients with RCC, respectively. As used in Tables 2 and 3, a gene is “correlated with” one class of patients if the average PBMC expression level of the gene in that class of patients is higher than that in the other class of patients. For instance, the average PBMC expression level of gene FLJ20420 in the class of patients who have less-than-365 TTD is higher than that in the class of patients who have greater-than-365 TTD (see Gene No. 1 in Table 2).

Each HG-U133A qualifier in Tables 2 and 3 represents an oligonucleotide probe set on the HG-U133A genechip. The RNA transcript(s) of a gene identified by a HG-U133A qualifier can hybridize under nucleic acid array hybridization conditions to at least one oligonucleotide probe (PM or perfect match probe) of that qualifier. Preferably, the RNA transcript(s) of the gene does not hybridize under nucleic acid array hybridization conditions to the mismatch probe (MM) of the PM probe. A mismatch probe is identical to the corresponding PM probe except for a single, homomeric substitution at or near the center of the mismatch probe. For a 25-mer PM probe, the MM probe has a homomeric base change at the 13th position.

In many cases, the RNA transcript(s) of a gene identified by a HG-U133A qualifier can hybridize under nucleic acid array hybridization conditions to at least 50%, 60%, 70%, 80%, 90% or 100% of the PM probes of the qualifier, but not to the corresponding mismatch probes of these PM probes. In many other cases, the discrimination score (R) for each of these PM probes, as measured by the ratio of the hybridization intensity difference of the corresponding probe pair (i.e., PM−MM) over the overall hybridization intensity (i.e., PM+MM), is no less than 0.015, 0.02, 0.05, 0.1, 0.2, 0.3, 0.4, 0.5 or greater. In one example, the RNA transcript(s) of the gene, when hybridized to the HG-U133A genechip according to the manufacturer's instructions, produces a “present” call under the default settings, i.e., the threshold Tau is 0.015 and the significance level α₁ is 0.4. See GeneChip® Expression Analysis—Data Analysis Fundamentals (Part No. 701190 Rev. 2, Affymetrix, Inc., 2002), the entire content of which is incorporated herein by reference.

The sequence of each PM probe on the HG-U133A genechip, and the corresponding target sequence from which the PM probe is derived, can be obtained from Affymetrix's sequence databases. See, for example, www.affymetrix.com/support/technical/byproduct.affx?product=hgu133. All of the PM probe sequences and the corresponding target sequences on the HG-U133A genechip are incorporated herein by reference.

Each gene listed in Tables 2 and 3, and the corresponding unigene ID(s), are identified according to HG-U133A genechip annotation. A unigene is composed of a non-redundant set of gene-oriented clusters. Each unigene cluster is believed to include sequences that represent a unique gene. Information for the genes listed in Tables 2 and 3 and their corresponding unigenes can also be obtained from the Entrez Gene and Unigene databases at National Center for Biotechnology Information (NCBI), Bethesda, Md.

In addition to Affymetrix annotations, gene(s) represented by a HG-U133A qualifier can also be identified by BLAST searching the target sequence of the qualifier against a human genome sequence database. Human genome sequence databases suitable for this purpose include, but are not limited to, the NCBI human genome database. NCBI provides BLAST programs, such as “blastn,” for searching its sequence databases. In one embodiment, the BLAST search of the NCBI human genome database is performed by using an unambiguous segment (e.g., the longest unambiguous segment) of the target sequence of a qualifier. Gene(s) represented by the qualifier is identified as those that have significant sequence identity to the unambiguous segment. In many cases, the identified gene(s) has at least 95%, 96%, 97%, 98%, 99%, or more sequence identity to the unambiguous segment.

As used herein, genes identified by the qualifiers in Tables 2 and 3 encompass not only those that are explicitly described therein, but also those that are not listed in the tables but nonetheless are capable of hybridizing to the PM probes of the qualifiers in the tables. All of these genes can be used as biological markers for the prognosis of RCC or other solid tumors.

Genes or classifiers that are prognostic or predictive of other clinically relevant stratifications can be similarly identified by using HG-U133A and the nearest neighbors analysis or another supervised or unsupervised clustering/learning algorithm. Likewise, the prediction accuracy, sensitivity or specificity for each classifier thus identified can be evaluated by leave-one-out cross validation or k-fold cross validation. In one example, a k-nearest-neighbors algorithm (see, for example, Armstrong, et al., Nature Genetics, 30:41-47 (2002)) is employed for selecting and evaluating gene classifiers. As used herein, “sensitivity” refers to the ratio of correct positive calls over the total of true positive calls plus false negative calls, and “specificity” refers to the ratio of correct negative calls over the total of true negative calls plus false positive calls.

As appreciated by one of ordinary skill in the art, genes that are prognostic or predictive of clinical stratifications of patients having other solid tumors can be similarly identified according to the present invention. The peripheral blood expression levels of these genes are correlated with clinical outcome of these patients.

### III. Prognosis of RCC or Other Solid Tumors

The prognosis genes of the present invention can be used as surrogate markers for the prognosis of RCC or other solid tumors. The prognosis genes can also be used for the selection of favorable treatments for patients with RCC or other solid tumors.

Any solid tumor or its treatment can be evaluated according to the present invention. Clinical outcome can be measured by a variety of clinical criteria, including but not limited to, TTP (e.g., less than or greater than a specified period), TTD (e.g., less than or greater than a specified period), progressive disease, non-progressive disease, stable disease, complete response, partial response, minor response, or a combination thereof. Non-responsiveness to a therapeutic treatment is also considered a measurable outcome.

Outcome prediction typically involves comparison of the peripheral blood expression profile of one or more prognosis genes in a solid tumor patient of interest (e.g., an RCC patient) to at least one reference expression profile. Each prognosis gene employed in the outcome prediction is differentially expressed in peripheral blood samples of solid tumor patients who have different clinical outcomes. These solid tumor patients have the same solid tumor as the patient of interest.

In one embodiment, the prognosis genes employed for the outcome prediction are selected such that the peripheral blood expression profile of each prognosis gene, as measured by Affymetrix HG-U133A genechips, is correlated with a class distinction under a class-based correlation analysis (such as the nearest-neighbor analysis or the SAM method), where the class distinction represents an idealized expression pattern of the selected genes in peripheral blood samples of solid tumor patients who have different clinical outcomes. In many cases, the selected prognosis genes are correlated with the class distinction at above the 50%, 25%, 10%, 5%, or 1% significance level under a random permutation test.

The prognosis genes can also be selected such that the average expression profile of each prognosis gene in peripheral blood samples of one class of solid tumor patients, as measured by Affymetrix HG-U133A genechips, is statistically different from that in another class of solid tumor patients. Both classes of patients have the same solid tumor (e.g., RCC) as the patient of interest. For instance, the p-value under a Student's t-test for the observed difference can be no more than 0.05, 0.01, 0.005, 0.001, or less. In addition, the prognosis genes can be selected such that the average peripheral blood expression level of each prognosis gene in one class of patients is at least 2-, 3-, 4-, 5-, 10-, or 20-fold different from that in another class of patients.

The expression profile of the patient of interest can be compared to one or more reference expression profiles. The reference expression profiles can be determined concurrently with the expression profile of the patient of interest. The reference expression profiles can also be predetermined or prerecorded in electronic or other types of storage media.

The reference expression profiles can include average expression profiles, or individual profiles representing peripheral blood gene expression patterns in particular patients. In one embodiment, the reference expression profiles include an average expression profile of the prognosis gene(s) in peripheral blood samples of reference patients who have the same solid tumor as the patient of interest and whose clinical outcome is known or determinable. Any averaging method may be used, such as arithmetic means, harmonic means, average of absolute values, average of log-transformed values, or weighted average. In one example, all of the reference patients have the same clinical outcome. In another example, the reference patients can be divided into at least two classes, each class of patients having a different respective clinical outcome. The average peripheral blood expression profile in each class of patients constitutes a separate reference expression profile, and the expression profile of the patient of interest is compared to each of these reference expression profiles.

In another embodiment, the reference expression profiles includes a plurality of expression profiles, each of which represents the peripheral blood expression pattern of the prognosis gene(s) in a particular patient who has the same solid tumor as the patient of interest and whose clinical outcome is known or determinable. Other types of reference expression profiles can also be used in the present invention.

The expression profile of the patient of interest and the reference expression profile(s) can be constructed in any form. In one embodiment, the expression profiles comprise the expression level of each prognosis gene used in outcome prediction. The expression levels can be absolute, normalized, or relative levels. Suitable normalization procedures include, but are not limited to, those used in nucleic acid array gene expression analyses or those described in Hill, et al., GENOME BIOL, 2:research0055.1-0055.13 (2001). In one example, the expression levels are normalized such that the mean is zero and the standard deviation is one. In another example, the expression levels are normalized based on internal or external controls, as appreciated by those skilled in the art. In still another example, the expression levels are normalized against one or more control transcripts with known abundances in blood samples. In many cases, the expression profile of the patient of interest and the reference expression profile(s) are constructed using the same or comparable methodologies.

In another embodiment, each expression profile being compared comprises one or more ratios between the expression levels of different prognosis genes. An expression profile can also include other measures that are capable of representing gene expression patterns.

The peripheral blood samples used in the present invention can be either whole blood samples, or samples comprising enriched PBMCS. In one example, the peripheral blood samples used for preparing the reference expression profile(s) comprise enriched or purified PBMCS, and the peripheral blood sample used for preparing the expression profile of the patient of interest is a whole blood sample. In another example, all of the peripheral blood samples employed in outcome prediction comprise enriched or purified PBMCS. In many cases, the peripheral blood samples are prepared from the patient of interest and reference patients using the same or comparable procedures.

Other types of blood samples can also be employed in the present invention, provided that a statistically significant correlation exists between patient outcome and the gene expression profiles in these blood samples.

The peripheral blood samples used in the present invention can be isolated from respective patients at any disease or treatment stage, provided that the correlation between the gene expression patterns in these peripheral blood samples and clinical outcome is statistically significant. In many embodiments, clinical outcome is measured by patients' response to a therapeutic treatment, and all of the blood samples used in outcome prediction are isolated prior to the therapeutic treatment. The expression profiles derived from these blood samples are therefore baseline expression profiles for the therapeutic treatment.

Construction of the expression profiles typically involves detection of the expression level of each prognosis gene used in the outcome prediction. Numerous methods are available for this purpose. For instance, the expression level of a gene can be determined by measuring the level of the RNA transcript(s) of the gene. Suitable methods include, but are not limited to, quantitative RT-PCT, Northern Blot, in situ hybridization, slot-blotting, nuclease protection assay, and nucleic acid array (including bead array). The expression level of a gene can also be determined by measuring the level of the polypeptide(s) encoded by the gene. Suitable methods include, but are not limited to, immunoassays (such as ELISA, RIA, FACS, or Western Blot), 2-dimensional gel electrophoresis, mass spectrometry, or protein arrays.

In one aspect, the expression level of a prognosis gene is determined by measuring the RNA transcript level of the gene in a peripheral blood sample. RNA can be isolated from the peripheral blood sample using a variety of methods. Exemplary methods include guanidine isothiocyanate/acidic phenol method, the TRIZOL® Reagent (Invitrogen), or the Micro-FastTrack™ 2.0 or FastTrack™ 2.0 mRNA Isolation Kits (Invitrogen). The isolated RNA can be either total RNA or mRNA. The isolated RNA can be amplified to cDNA or cRNA before subsequent detection or quantitation. The amplification can be either specific or non-specific. Suitable amplification methods include, but are not limited to, reverse transcriptase PCR (RT-PCR), isothermal amplification, ligase chain reaction, and Qbeta replicase.

In one embodiment, the amplification protocol employs reverse transcriptase. The isolated mRNA can be reverse transcribed into cDNA using a reverse transcriptase, and a primer consisting of oligo d(T) and a sequence encoding the phage T7 promoter. The cDNA thus produced is single-stranded. The second strand of the cDNA is synthesized using a DNA polymerase, combined with an RNase to break up the DNA/RNA hybrid. After synthesis of the double-stranded cDNA, T7 RNA polymerase is added, and cRNA is then transcribed from the second strand of the doubled-stranded cDNA. The amplified cDNA or cRNA can be detected or quantitated by hybridization to labeled probes. The cDNA or cRNA can also be labeled during the amplification process and then detected or quantitated.

In another embodiment, quantitative RT-PCR (such as TaqMan, ABI) is used for detecting or comparing the RNA transcript level of a prognosis gene of interest. Quantitative RT-PCR involves reverse transcription (RT) of RNA to cDNA followed by relative quantitative PCR (RT-PCR).

In PCR, the number of molecules of the amplified target DNA increases by a factor approaching two with every cycle of the reaction until some reagent becomes limiting. Thereafter, the rate of amplification becomes increasingly diminished until there is not an increase in the amplified target between cycles. If a graph is plotted on which the cycle number is on the X axis and the log of the concentration of the amplified target DNA is on the Y axis, a curved line of characteristic shape can be formed by connecting the plotted points. Beginning with the first cycle, the slope of the line is positive and constant. This is said to be the linear portion of the curve. After some reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR is proportional to the starting concentration of the target before the PCR is begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction.

The final concentration of the target DNA in the plateau portion of the curve is determined by the availability of reagents in the reaction mix and is independent of the original concentration of target DNA. Therefore, in one embodiment, the sampling and quantifying of the amplified PCR products are carried out when the PCR reactions are in the linear portion of their curves. In addition, relative concentrations of the amplifiable cDNAs can be normalized to some independent standard, which may be based on either internally existing RNA species or externally introduced RNA species. The abundance of a particular mRNA species may also be determined relative to the average abundance of all mRNA species in the sample.

In one embodiment, the PCR amplification utilizes internal PCR standards that are approximately as abundant as the target. This strategy is effective if the products of the PCR amplifications are sampled during their linear phases. If the products are sampled when the reactions are approaching the plateau phase, then the less abundant product may become relatively over-represented. Comparisons of relative abundances made for many different RNA samples, such as is the case when examining RNA samples for differential expression, may become distorted in such a way as to make differences in relative abundances of RNAs appear less than they actually are. This can be improved if the internal standard is much more abundant than the target. If the internal standard is more abundant than the target, then direct linear comparisons may be made between RNA samples.

A problem inherent in clinical samples is that they are of variable quantity or quality. This problem can be overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs. While empirical determination of the linear range of the amplification curve and normalization of cDNA preparations are tedious and time-consuming processes, the resulting RT-PCR assays may, in certain cases, be superior to those derived from a relative quantitative RT-PCR with an internal standard.

In yet another embodiment, nucleic acid arrays (including bead arrays) are used for detecting or comparing the expression profiles of a prognosis gene of interest. The nucleic acid arrays can be commercial oligonucleotide or cDNA arrays. They can also be custom arrays comprising concentrated probes for the prognosis genes of the present invention. In many examples, at least 15%, 20%, 25%, 30%, 35%, 40%, 45%, 50%, or more of the total probes on a custom array of the present invention are probes for RCC or other solid tumor prognosis genes. These probes can hybridize under stringent or nucleic acid array hybridization conditions to the RNA transcripts, or the complements thereof, of the corresponding prognosis genes.

As used herein, “stringent conditions” are at least as stringent as, for example, conditions G-L shown in Table 5. “Highly stringent conditions” are at least as stringent as conditions A-F shown in Table 5. Hybridization is carried out under the hybridization conditions (Hybridization Temperature and Buffer) for about four hours, followed by two 20-minute washes under the corresponding wash conditions (Wash Temp. and Buffer).

TABLE 5
Stringency Conditions

| Stringency | Polynucleotide | Hybrid | Hybridization | Wash Temp. |
|---|---|---|---|---|
| Condition | Hybrid | Length (bp)¹ | Temperature and Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{H}SSPE (1x SSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers. | | | | |
| T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs | | | | |
| # in length, Tₘ(° C.) = 81.5 + 16.6(log₁₀[Na⁺]) + 0.41(% G + C) − (600/N), where N is the number of bases in the hybrid, and [Na⁺] is the molar concentration of sodium ions in the hybridization buffer ([Na⁺] for 1xSSC = 0.165 M). | | | | |

In one example, a nucleic acid array of the present invention includes at least 2, 5, 10, or more different probes. Each of these probes is capable of hybridizing under stringent or nucleic acid array hybridization conditions to a different respective prognosis gene of the present invention. Multiple probes for the same prognosis gene can be used on the same nucleic acid array. The probe density on the array can be in any range.

The probes for a prognosis gene of the present invention can be DNA, RNA, PNA, or a modified form thereof. The nucleotide residues in each probe can be either naturally occurring residues (such as deoxyadenylate, deoxycytidylate, deoxyguanylate, deoxythymidylate, adenylate, cytidylate, guanylate, and uridylate), or synthetically produced analogs that are capable of forming desired base-pair relationships. Examples of these analogs include, but are not limited to, aza and deaza pyrimidine analogs, aza and deaza purine analogs, and other heterocyclic base analogs, wherein one or more of the carbon and nitrogen atoms of the purine and pyrimidine rings are substituted by heteroatoms, such as oxygen, sulfur, selenium, and phosphorus. Similarly, the polynucleotide backbones of the probes can be either naturally occurring (such as through 5′ to 3′ linkage), or modified. For instance, the nucleotide units can be connected via non-typical linkage, such as 5′ to 2′ linkage, so long as the linkage does not interfere with hybridization. For another instance, peptide nucleic acids, in which the constitute bases are joined by peptide bonds rather than phosphodiester linkages, can be used.

The probes for the prognosis genes can be stably attached to discrete regions on a nucleic acid array. By “stably attached,” it means that a probe maintains its position relative to the attached discrete region during hybridization and signal detection. The position of each discrete region on the nucleic acid array can be either known or determinable. All of the methods known in the art can be used to make the nucleic acid arrays of the present invention.

In another embodiment, nuclease protection assays are used to quantitate RNA transcript levels in peripheral blood samples. There are many different versions of nuclease protection assays. The common characteristic of these nuclease protection assays is that they involve hybridization of an antisense nucleic acid with the RNA to be quantified. The resulting hybrid double-stranded molecule is then digested with a nuclease that digests single-stranded nucleic acids more efficiently than double-stranded molecules. The amount of antisense nucleic acid that survives digestion is a measure of the amount of the target RNA species to be quantified. Examples of suitable nuclease protection assays include the RNase protection assay provided by Ambion, Inc. (Austin, Tex.).

Hybridization probes or amplification primers for the prognosis genes of the present invention can be prepared by using any method known in the art. For prognosis genes whose genomic locations have not been determined or whose identities are solely based on EST or mRNA data, the probes/primers for these genes can be derived from the target sequences of the corresponding qualifiers, or the corresponding EST or mRNA sequences.

In one embodiment, the probes/primers for a prognosis gene significantly diverge from the sequences of other prognosis genes. This can be achieved by checking potential probe/primer sequences against a human genome sequence database, such as the Entrez database at the NCBI. One algorithm suitable for this purpose is the BLAST algorithm. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. The initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always >0) and N (penalty score for mismatching residues; always <0). The BLAST algorithm parameters W, T, and X determine the sensitivity and speed of the alignment. These parameters can be adjusted for different purposes, as appreciated by those skilled in the art.

In another aspect, the expression levels of the prognosis genes of the present invention are determined by measuring the levels of polypeptides encoded by the prognosis genes. Methods suitable for this purpose include, but are not limited to, immunoassays such as ELISA, RIA, FACS, dot blot, Western Blot, immunohistochemistry, and antibody-based radioimaging. In addition, high-throughput protein sequencing, 2-dimensional SDS-polyacrylamide gel electrophoresis, mass spectrometry, or protein arrays can be used.

In one embodiment, ELISAs are used for detecting the levels of the target proteins. In an exemplifying ELISA, antibodies capable of binding to the target proteins are immobilized onto selected surfaces exhibiting protein affinity, such as wells in a polystyrene or polyvinylchloride microtiter plate. Samples to be tested are then added to the wells. After binding and washing to remove non-specifically bound immunocomplexes, the bound antigen(s) can be detected. Detection can be achieved by the addition of a second antibody which is specific for the target proteins and is linked to a detectable label. Detection can also be achieved by the addition of a second antibody, followed by the addition of a third antibody that has binding affinity for the second antibody, with the third antibody being linked to a detectable label. Before being added to the microtiter plate, cells in the samples can be lysed or extracted to separate the target proteins from potentially interfering substances.

In another exemplifying ELISA, the samples suspected of containing the target proteins are immobilized onto the well surface and then contacted with the antibodies. After binding and washing to remove non-specifically bound immunocomplexes, the bound antigen is detected. Where the initial antibodies are linked to a detectable label, the immunocomplexes can be detected directly. The immunocomplexes can also be detected using a second antibody that has binding affinity for the first antibody, with the second antibody being linked to a detectable label.

Another exemplary ELISA involves the use of antibody competition in the detection. In this ELISA, the target proteins are immobilized on the well surface. The labeled antibodies are added to the well, allowed to bind to the target proteins, and detected by means of their labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test samples. Examples of these nonspecific proteins include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can be used. After binding of a protein or antibody to the well, coating with a non-reactive material to reduce background, and washing to remove unbound material, the immobilizing surface is contacted with the control or clinical or biological sample to be tested under conditions effective to allow immunocomplex (antigen/antibody) formation. These conditions may include, for example, diluting the antigens and antibodies with solutions such as BSA, bovine gamma globulin (BGG) and phosphate buffered saline (PBS)/Tween and incubating the antibodies and antigens at room temperature for about 1 to 4 hours or at 4° C. overnight. Detection of the immunocomplex is facilitated by using a labeled secondary binding ligand or antibody, or a secondary binding ligand or antibody in conjunction with a labeled tertiary antibody or third binding ligand.

Following all incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detecting means, the second or third antibody can have an associated label to allow detection. In one embodiment, the label is an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation (e.g., incubation for 2 hours at room temperature in a PBS-containing solution such as PBS-Tween).

After incubation with the labeled antibody, and subsequent washing to remove unbound material, the amount of label can be quantified, e.g., by incubation with a chromogenic substrate such as urea and bromocresol purple or 2,2′-azido-di-(3-ethyl)-benzthiazoline-6-sulfonic acid (ABTS) and H₂O₂, in the case of peroxidase as the enzyme label. Quantitation can be achieved by measuring the degree of color generation, e.g., using a spectrophotometer.

Another method suitable for detecting polypeptide levels is RIA (radioimmunoassay). An exemplary RIA is based on the competition between radiolabeled-polypeptides and unlabeled polypeptides for binding to a limited quantity of antibodies. Suitable radiolabels include, but are not limited to, I¹²⁵. In one embodiment, a fixed concentration of I¹²⁵-labeled polypeptide is incubated with a series of dilution of an antibody specific to the polypeptide. When the unlabeled polypeptide is added to the system, the amount of the I¹²⁵-polypeptide that binds to the antibody is decreased. A standard curve can therefore be constructed to represent the amount of antibody-bound I¹²⁵-polypeptide as a function of the concentration of the unlabeled polypeptide. From this standard curve, the concentration of the polypeptide in unknown samples can be determined. Protocols for conducting RIA are well known in the art.

Suitable antibodies for the present invention include, but are not limited to, polyclonal antibodies, monoclonal antibodies, chimeric antibodies, humanized antibodies, single chain antibodies, Fab fragments, or fragments produced by a Fab expression library. Neutralizing antibodies (i.e., those which inhibit dimer formation) can also be used. Methods for preparing these antibodies are well known in the art. In one embodiment, the antibodies of the present invention can bind to the corresponding prognosis gene products or other desired antigens with binding affinities of at least 10⁴ M⁻¹, 10⁵ M⁻¹, 10⁶ M⁻¹, 10⁷ M⁻¹, or more.

The antibodies of the present invention can be labeled with one or more detectable moieties to allow for detection of antibody-antigen complexes. The detectable moieties can include compositions detectable by spectroscopic, enzymatic, photochemical, biochemical, bioelectronic, immunochemical, electrical, optical or chemical means. The detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

The antibodies of the present invention can be used as probes to construct protein arrays for the detection of expression profiles of the prognosis genes. Methods for making protein arrays or biochips are well known in the art. In many embodiments, a substantial portion of probes on a protein array of the present invention are antibodies specific for the prognosis gene products. For instance, at least 10%, 20%, 30%, 40%, 50%, or more probes on the protein array can be antibodies specific for the prognosis gene products.

In yet another aspect, the expression levels of the prognosis genes are determined by measuring the biological functions or activities of these genes. Where a biological function or activity of a gene is known, suitable in vitro or in vivo assays can be developed to evaluate the function or activity. These assays can be subsequently used to assess the level of expression of the prognosis gene.

After the expression level of each prognosis gene is determined, numerous approaches can be employed to compare expression profiles. Comparison of the expression profile of a patient of interest to the reference expression profile(s) can be conducted manually or electronically. In one example, comparison is carried out by comparing each component in one expression profile to the corresponding component in a reference expression profile. The component can be the expression level of a prognosis gene, a ratio between the expression levels of two prognosis genes, or another measure capable of representing gene expression patterns. The expression level of a gene can have an absolute or a normalized or relative value. The difference between two corresponding components can be assessed by fold changes, absolute differences, or other suitable means.

Comparison of the expression profile of a patient of interest to the reference expression profile(s) can also be conducted using pattern recognition or comparison programs, such as the k-nearest-neighbors algorithm as described in Armstrong, et al., NATURE GENETICS, 30:41-47 (2002), or the weighted voting algorithm as described below. In addition, the serial analysis of gene expression (SAGE) technology, the GEMTOOLS gene expression analysis program (Incyte Pharmaceuticals), the GeneCalling and Quantitative Expression Analysis technology (Curagen), and other suitable methods, programs or systems can be used to compare expression profiles.

Multiple prognosis genes can be used in the comparison of expression profiles. For instance, 2, 4, 6, 8, 10, 12, 14, or more prognosis genes can be used. In addition, the prognosis gene(s) used in the comparison can be selected to have relatively small p-values (e.g., two-sided p-values). In many examples, the p-values indicate the statistical significance of the difference between gene expression levels in different classes of patients. In many other examples, the p-values suggest the statistical significance of the correlation between gene expression patterns and clinical outcome. In one embodiment, the prognosis genes used in the comparison have p-values of no greater than 0.05, 0.01, 0.001, 0.0005, 0.0001, or less. Prognosis genes with p-values of greater than 0.05 can also be used. These genes may be identified, for instance, by using a relatively small number of blood samples.

Similarity or difference between the expression profile of a patient of interest and a reference expression profile is indicative of the class membership of the patient of interest. Similarity or difference can be determined by any suitable means. The comparison can be qualitative, quantitative, or both.

In one example, a component in a reference profile is a mean value, and the corresponding component in the expression profile of the patient of interest falls within the standard deviation of the mean value. In such a case, the expression profile of the patient of interest may be considered similar to the reference profile with respect to that particular component. Other criteria, such as a multiple or fraction of the standard deviation or a certain degree of percentage increase or decrease, can be used to measure similarity.

In another example, at least 50% (e.g., at least 60%, 70%, 80%, 90%, or more) of the components in the expression profile of the patient of interest are considered similar to the corresponding components in a reference profile. Under these circumstances, the expression profile of the patient of interest may be considered similar to the reference profile. Different components in the expression profile may have different weights for the comparison. In some cases, lower percentage thresholds (e.g., less than 50% of the total components) are used to determine similarity.

The prognosis gene(s) and the similarity criteria can be selected such that the accuracy of outcome prediction (the ratio of correct calls over the total of correct and incorrect calls) is relatively high. For instance, the accuracy of prediction can be at least 50%, 60%, 70%, 80%, 90%, or more. Prognosis genes with prediction accuracy of less than 50% can also be used, provided that the predictions are statistically significant.

The effectiveness of outcome prediction can also be assessed by sensitivity and specificity. The prognosis genes and the comparison criteria can be selected such that both the sensitivity and specificity of outcome prediction are relatively high. For instance, the sensitivity and specificity of the prognosis gene(s) or classifier employed can be at least 50%, 60%, 70%, 80%, 90%, 95%, or more. Prognosis genes or classifiers having sensitivities or specificities of less than 50% can also be used, provided that the predictions are statistically significant.

Moreover, peripheral blood expression profile-based outcome prediction can be combined with other clinical evidence or prognostic methods to improve the effectiveness or accuracy of outcome prediction.

In many embodiments, the expression profile of a patient of interest is compared to at least two reference expression profiles. Each reference expression profile can include an average expression profile, or a set of individual expression profiles each of which represents the peripheral blood gene expression pattern in a particular solid tumor (e.g., RCC) patient or disease-free human. Suitable methods for comparing one expression profile to two or more reference expression profiles include, but are not limited to, the weighted voting algorithm or the k-nearest-neighbors algorithm. Softwares capable of performing these algorithms include, but are not limited to, GeneCluster 2 software. GeneCluster 2 software is available from MIT Center for Genome Research at Whitehead Institute (e.g., www-genome.wi.mit.edu/cancer/software/genecluster2/gc2.html).

Both the weighted voting and k-nearest-neighbors algorithms employ gene classifiers that can effectively assign a patient of interest to an outcome class. By “effectively,” it means that the class assignment is statistically significant. In one example, the effectiveness of class assignment is evaluated by leave-one-out cross validation or k-fold cross validation. The prediction accuracy under these cross validation methods can be, for instance, at least 50%, 60%, 70%, 80%, 90%, 95%, or more. The prediction sensitivity or specificity under these cross validation methods can also be at least 50%, 60%, 70%, 80%, 90%, 95%, or more. Prognosis genes or class predictors with low assignment sensitivity/specificity or low cross validation accuracy, such as less than 50%, can also be used in the present invention.

Under one version of the weighted voting algorithm, each gene in a class predictor casts a weighted vote for one of the two classes (class 0 and class 1). The vote of gene “g” can be defined as v_{g}=a_{g}(x_{g}-b_{g}), wherein a_{g} equals to P(g,c) and reflects the correlation between the expression level of gene “g” and the class distinction between the two classes, b_{g} is calculated as b_{g}=[x0(g)+x1(g)]/2 and represents the average of the mean logs of the expression levels of gene “g” in class 0 and class 1, and x_{g} is the normalized log of the expression level of gene “g” in the sample of interest. A positive v_{g} indicates a vote for class 0, and a negative v_{g} indicates a vote for class 1. V0 denotes the sum of all positive votes, and V1 denotes the absolute value of the sum of all negative votes. A prediction strength PS is defined as PS=(V0−V1)/(V0+V1). Thus, the prediction strength varies between −1 and 1 and can indicate the support for one class (e.g., positive PS) or the other (e.g., negative PS). A prediction strength near “0” suggests narrow margin of victory, and a prediction strength close to “1” or “−1” indicates wide margin of victory. See Slonim, et al., PROCS. OF THE FOURTH ANNUAL INTERNATIONAL CONFERENCE ON COMPUTATIONAL MOLECULAR BIOLOGY, Tokyo, Japan, April 8-11, p263-272 (2000); and Golub, et al., SCIENCE, 286: 531-537 (1999).

Suitable prediction strength (PS) thresholds can be assessed by plotting the cumulative cross-validation error rate against the prediction strength. In one embodiment, a positive predication is made if the absolute value of PS for the sample of interest is no less than 0.3. Other PS thresholds, such as no less than 0.1, 0.2, 0.4 or 0.5, can also be selected for class prediction. In many embodiments, a threshold is selected such that the accuracy of prediction is optimized and the incidence of both false positive and false negative results is minimized.

Any class predictor constructed according to the present invention can be used for the class assignment of a solid tumor patient of interest (e.g., an RCC patient). In many examples, a class predictor employed in the present invention includes n prognosis genes identified by the neighborhood analysis, where n is an integer greater than 1. A half of these prognosis genes has the largest P(g,c) scores, and the other half has the largest −P(g,c) scores. The number n therefore is the only free parameter in defining the class predictor.

The expression profile of a patient of interest can also be compared to two or more reference expression profiles by other means. For instance, the reference expression profiles can include an average peripheral blood expression profile for each class of patients. The fact that the expression profile of a patient of interest is more similar to one reference profile than to another suggests that the patient of interest is more likely to have the clinical outcome associated with the former reference profile than that associated with the latter reference profile.

In one particular embodiment, the present invention features prediction of clinical outcome of an RCC patient of interest. Prognosis genes or classifiers suitable for this purpose include, but are not limited to, those described in Tables 2, 3 or 4.

In one example, RCC patients can be divided into at least two classes based on their TTD in response to a therapeutic treatment (e.g., a CCI-779 therapy). A first class of patients has a first specified TTD (e.g., TTD of less than 365 days from initiation of the therapeutic treatment), and a second class of patients has a second specified TTD (e.g., TTD of more than 365 days from initiation of the therapeutic treatment). Genes that are substantially correlated with the class distinction between these two classes of patients can be identified and used to predict the class membership of an RCC patient of interest. In many cases, all of the expression profiles used in the outcome prediction are baseline profiles prepared from peripheral blood samples isolated prior to the therapeutic treatment. Examples of RCC prognosis genes suitable for this purpose include those selected from Table 2, and examples of suitable classifiers include classifiers 1-7 in Table 4. The present invention contemplates the use of any combination of Gene Nos. 1-14 of Table 2 for prediction of clinical outcome of an RCC patient of interest. Methods suitable for this purpose include, but are not limited to, RT-PCR, ELISA, functional assays, or pattern recognition programs (e.g., the weighted voting or k-nearest-neighbors algorithms).

In another example, a first class of RCC patients has a specified TTP (e.g., TTP of no less than 106 days from initiation of a therapeutic treatment, such as a CCI-779 therapy), and a second class of patients has another specified TTP (e.g., TTP of less than 106 days from initiation of the therapeutic treatment). Prognosis genes capable of assigning an RCC patient of interest to one of the above two outcome classes include, but are not limited to, those depicted in Table 3, and suitable classifiers include classifiers 8-21 in Table 4. The present invention contemplates the use of any combination of Gene Nos. 1-28 of Table 3 for prediction of clinical outcome of an RCC patient of interest. Methods suitable for this purpose include, but are not limited to, RT-PCR, ELISA, functional assays, or pattern recognition programs (e.g., the weighted voting or k-nearest-neighbors algorithms).

In a further example, the expression profile of an RCC patient of interest is compared to two or more reference expression profiles by using a weighted voting or k-nearest-neighbors algorithm and a classifier selected from Table 4.

Prognosis genes or class predictors capable of distinguishing three or more outcome classes can also be employed in the present invention. These prognosis genes can be identified using multi-class correlation metrics. Suitable programs for carrying out multi-class correlation analysis include, but are not limited to, GeneCluster 2 software (MIT Center for Genome Research at Whitehead Institute, Cambridge, Mass.). Under the analysis, patients having a specified solid tumor (e.g., RCC) are divided into at least three classes, and each class of patients has a different respective clinical outcome. The prognosis genes identified under multi-class correlation analysis are differentially expressed in PBMCs of one class of patients relative to PBMCs of other classes of patients. In one embodiment, the identified prognosis genes are correlated with a class distinction at above the 1%, 5%, 10%, 25%, or 50% significance level under a permutation test. The class distinction represents an idealized expression pattern of the identified genes in peripheral blood samples of patients who have different clinical outcomes.

The present invention also features electronic systems useful for the prognosis or selection of treatment of RCC and other solid tumors. These systems include input or communication devices for receiving the expression profile of an RCC patient of interest as well as the reference expression profile(s). The reference expression profile(s) can be stored in a database or another medium. The comparison between expression profiles can be conduced electronically, such as through a processor or a computer. The processor or computer can execute one or more programs to compare the expression profile of the patient of interest to the reference expression profile(s). The program(s) can be stored in a memory or downloaded from another source, such as an internet server. In one example, the program(s) includes a k-nearest-neighbors or weighted voting algorithm. In another example, the electronic system is coupled to a nucleic acid array and can receive or process expression data generated by the nucleic acid array.

In still another aspect, the present invention provides kits useful for the prognosis or selection of treatment of RCC or other solid tumors. Each kit includes at least one probe for an RCC or solid tumor prognosis gene (e.g., a gene selected from Tables 2 or 3). Any type of probe can be using in the present invention, such as hybridization probes, amplification primers, or antibodies.

In one embodiment, a kit of the present invention includes at least 1, 2, 3, 4, 5, 6, 7, 8, 9, 10, or more polynucleotide probes or primers. Each probe/primer can hybridize under stringent or nucleic acid array hybridization conditions to a different respective RCC or solid tumor prognosis gene, such as those selected from Tables 2 or 3. In one example, a kit of the present invention includes probes capable of hybridizing under stringent or nucleic acid array hybridization conditions to the respective genes in a classifier of the present invention, such as those selected from Table 4. As used herein, a polynucleotide can hybridize to a gene if the polynucleotide can hybridize to an RNA transcript, or the complement thereof, of the gene.

In another embodiment, a kit of the present invention includes one or more antibodies, each of which is capable of binding to a polypeptide encoded by a different respective RCC or solid tumor prognosis gene, such as those selected from Tables 2 or 3. In one example, a kit of the present invention includes antibodies capable of binding to the respective polypeptides encoded by the genes in a classifier of the present invention, such as those selected from Table 4.

The probes employed in the present invention can be either labeled or unlabeled. Labeled probes can be detectable by spectroscopic, photochemical, biochemical, bioelectronic, immunochemical, electrical, optical, chemical, or other suitable means. Exemplary labeling moieties for a probe include radioisotopes, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like.

The kits of the present invention can also have containers containing buffer(s) or reporter-means. In addition, the kits can include reagents for conducting positive or negative controls. In one embodiment, the probes employed in the present invention are stably attached to one or more substrate supports. Nucleic acid hybridization or immunoassays can be directly carried out on the substrate support(s). Suitable substrate supports for this purpose include, but are not limited to, glasses, silica, ceramics, nylons, quartz wafers, gels, metals, papers, beads, tubes, fibers, films, membranes, column matrixes, or microtiter plate wells.

### IV. Selection of Treatment of RCC and Other Solid Tumors

The present invention allows for personalized treatment of RCC or other solid tumors. Clinical outcome of a patient of interest can be predicted according to the present invention before any treatment. A good prognosis of the patient indicates that the treatment is likely to be effective, while a poor prognosis suggests that a different therapy may be more suitable for the patient. This pre-treatment analysis helps patients avoid unnecessary adverse reactions and provides improved safety and increased benefit/risk ratio for the treatment.

In one embodiment, the prognosis of an RCC patient of interest is evaluated before any treatment with CCI-779. Prognosis genes suitable for this purpose include, but are not limited to, those depicted in Tables 2 or 3. Any prognosis method described herein can be used, such as RT-PCR, ELISA, protein functional assays, or patent recognition programs (such as the k-nearest-neighbors or weighted voting algorithms). A good prognosis indicates suitability of CCI-779 treatment for the RCC patient of interest. Good versus poor prognosis can be measured by TTD (e.g., greater than one year versus less than one year) or TTP (e.g., greater than three months versus less than three months). Other measures can also be used to determine good or poor prognosis.

The present invention also features the selection of favorable treatment(s) for a patient of interest. Numerous treatment options or regimes can be analyzed by the present invention. Prognosis genes for each treatment can be identified. The peripheral blood expression profiles of these prognosis genes in a patient of interest are indicative of the clinical outcome of the patient and, therefore, can be used as surrogate markers for the identification or selection of treatments that have favorable prognoses for the patient. As used herein, a “favorable” prognosis is a prognosis that is better than the prognoses of the majority of all other available treatments for the patient of interest. The treatment regime with the best prognosis can also be identified.

Any type of cancer treatment can be evaluated by the present invention. For instance, RCC can be treated by drug therapies. Suitable drugs include cytokines, such as interferon or interleukin 2, and chemotherapy drugs, such as CCI-779, AN-238, vinblastine, floxuridine, 5-fluorouracil, or tamoxifen. AN238 is a cytotoxic agent which has 2-pyrrolinodoxorubicin linked to a somatostatin (SST) carrier octapeptide. AN238 can be targeted to SST receptors on the surface of RCC tumor cells. Chemotherapy drugs can be used individually or in combination with other drugs, cytokines, or therapies. In addition, monoclonal antibodies, antiangiogenesis drugs, or anti-growth factor drugs can be employed to treat RCC.

RCC treatment can also be surgical. Suitable surgical choices include, but are not limited to, radical nephrectomy, partial nephrectomy, removal of metastases, arterial embolization, laparoscopic nephrectomy, cryoablation, and nephron-sparing surgery. Moreover, radiation, gene therapy, immunotherapy, adoptive immunotherapy, or any other conventional or experimental therapy can be used.

Treatment options for prostate cancer, head/neck cancer, and other solid tumors are known in the art. For instance, prostate cancer treatments include, but are not limited to, radiation therapy, hormonal therapy, and cryotherapy. The present invention also contemplates the use of prognosis genes for other novel or experimental treatments of solid tumors.

Treatment selection can be conducted manually or electronically. Reference expression profiles or gene classifiers can be stored in a database. Programs capable of performing algorithms such as the k-nearest-neighbors or weighted voting algorithms can be used to compare the peripheral blood expression profile of a patient of interest to the database to determine which treatment should be used for the patient.

Identification of prognosis gene may be affected by the disease stage of a solid tumor. For instance, prognosis genes can be identified from patients at a particular disease stage. Genes thus identified may be more effective in predicting clinical outcome of a patient of interest who is also at that disease stage.

Disease stages may also affect treatment selection. For instance, for RCC patients in stages I or II, radical or partial nephrectomy is commonly selected. For RCC patients in stage III, radical nephrectomy is among the preferred treatments. For RCC patients in stage IV, cytokine immunotherapy, combined immunotherapy and chemotherapy, or other drug therapies can be employed. Therefore, the disease stage of a patient of interest can be used to assist the gene expression-based selection for a favorable treatment of the patient.

It should be understood that the above-described embodiments and the following examples are given by way of illustration, not limitation. Various changes and modifications within the scope of the present invention will become apparent to those skilled in the art from the present description.

### V. EXAMPLES

### Example 1

### Purification of PBMCs and RNA

Whole blood was collected from RCC patients prior to initiation of CCI-779 therapy. The blood samples were drawn into CPT Cell Preparation Vacutainer Tubes (Becton Dickinson). For each sample, the target volume was 8 ml. PBMCs were isolated over Ficoll gradients according to the manufacturer's protocol (Becton Dickinson). PBMC pellets were stored at −80° C. until samples were processed for RNA.

RNA purification was performed using QIA shredders and Qiagen Rneasy® mini-kits. Samples were harvested in RLT lysis buffer (Qiagen, Valencia, Calif., USA) containing 0.1% beta-mercaptoethanol and processed for total RNA isolation using the RNeasy mini kit (Qiagen, Valencia, Calif., USA). Eluted RNA was quantified using a 96 well plate UV reader monitoring A260/280. RNA qualities (bands for 18S and 28S) were checked by agarose gel electrophoresis in 2% agarose gels. The remaining RNA was stored at −80° C. until processed for Affymetrix genechip hybridization

### Example 2

### RNA Amplification and Generation of GeneChip Hybridization Probes

Labeled target for oligonucleotide arrays was prepared using a modification of the procedure described in Lockhart, et al., NATURE BIOTECHNOLOGY, 14:1675-1680 (1996). Two micrograms of total RNA were converted to cDNA using an oligo-d(T)₂₄ primer containing a T7 DNA polymerase promoter at the 5′ end. The cDNA was used as the template for in vitro transcription using a T7 DNA polymerase kit (Ambion, Woodlands, Tex., USA) and biotinylated CTP and UTP (Enzo, Farmingdale, N.Y., USA). Labeled cRNA was fragmented in 40 mM Tris-acetate pH 8.0, 100 mM KOAc, 30 mM MgOAc for 35 min at 94° C. in a final volume of 40 mL. Ten micrograms of labeled target were diluted in 1×MES buffer with 100 mg/mL herring sperm DNA and 50 mg/mL acetylated BSA. To normalize arrays to each other and to estimate the sensitivity of the oligonucleotide arrays, in vitro synthesized transcripts of 11 bacterial genes were included in each hybridization reaction as described in Hill, et al., GENOME BIOL., 2:research0055.1-0055.13 (2001). The abundance of these transcripts ranged from 1:300000 (3 ppm) to 1:1000 (1000 ppm) stated in terms of the number of control transcripts per total transcripts. As determined by the signal response from these control transcripts, the sensitivity of detection of the arrays ranged between 2.33 and 4.5 copies per million.

Labeled sequences were denatured at 99° C. for 5 min and then 45° C. for 5 min and hybridized to oligonucleotide arrays comprised of a large number of human genes (HG-U95A or HG-U133A, Affymetrix, Santa Clara, Calif., USA). Arrays were hybridized for 16 h at 45° C. with rotation at 60 rpm. After hybridization, the hybridization mixtures were removed and stored, and the arrays were washed and stained with Streptavidin R-phycoerythrin (Molecular Probes) using GeneChip Fluidics Station 400 and scanned with a Hewlett Packard GeneArray Scanner following the manufacturer's instructions. These hybridization and wash conditions are collectively referred to as “nucleic acid array hybridization conditions.”

### Example 3

### Determination of Gene Expression Frequencies and Processing of Expression Data

Array images were processed using the Affymetrix MicroArray Suite software (MAS) such that raw array image data (.dat) files produced by the array scanner were reduced to probe feature-level intensity summaries (.cel files) using the desktop version of MAS. Using the Gene Expression Data System (GEDS) as a graphical user interface, users provide a sample description to the Expression Profiling Information and Knowledge System (EPIKS) Oracle database and associate the correct cel file with the description. The database processes then invoke the MAS software to create probeset summary values; probe intensities are summarized for each message using the Affymetrix Average Difference algorithm and the Affymetrix Absolute Detection metric (Absent, Present, or Marginal) for each probeset. MAS is also used for the first pass normalization by scaling the trimmed mean to a value of 100. The database processes also calculate a series of chip quality control metrics and store all the raw data and quality control calculations in the database.

Data analysis and absent/present call determination was performed on raw fluorescent intensity values using MAS software (Affymetrix). “Present” calls are calculated by MAS software by estimating whether a transcript is detected in a sample based on the strength of the gene's signal compared to background. The “average difference” values for each transcript were normalized to “frequency” values using the scaled frequency normalization method (Hill, et al., GENOME BIOL, 2:research0055.1-0055.13 (2001)) in which the average differences for 11 control cRNAs with known abundance spiked into each hybridization solution were used to generate a global calibration curve. This calibration was then used to convert average difference values for all transcripts to frequency estimates, stated in units of parts per million ranging from 1:300,000 (˜3 parts per million (ppm)) to 1:1000 (1000 ppm). The normalization refers the average difference values on each chip to a calibration curve constructed from the average difference values for the 11 control transcripts with known abundance that were spiked into each hybridization solution. In many instances, the normalization method utilizes a trimmed-mean normalization, followed by fitting of a pooled standard curve across all chips, which is used to compute “frequency” values and per-chip sensitivity estimates. The resulting metric is referred to as a scaled frequency and normalizes between all arrays.

Genes that did not have any relevant information were excluded from the data comparison. In comparisons of disease-free PBMCs with RCC PBMCs, this was accomplished using two data reduction filters: 1) any gene that was called Absent on all GeneChips (as determined by the Affymetrix Absolute Detection metric in MAS) was removed from the dataset; 2) any gene that was expressed at a normalized frequency of <10 ppm on all GeneChips was removed from the dataset to ensure that any gene kept in the analysis set was detected at a frequency of at least 10 ppm at least once. For some multivariate prediction analyses more stringent data reduction filters were used (25% P, and average frequency >5 ppm) in order to decrease the likelihood that low level or infrequently detected transcripts would be identified in gene classifiers.

### Example 4

### Pearson's-Based Assessment of Outlier Samples

To identify outlier samples, the square of the pairwise Pearson correlation coefficient (r2) among all pairs of samples was computed using Splus (Version 5.1). Specifically, the computation was started from the G×S matrix of expression values, where G is the total number of probesets and S is the total number of samples. r2-values between samples in this matrix were calculated. The result was a symmetric S×S matrix of r2-values. This matrix measures the similarity between each sample and all other samples in the analysis. Since all of these samples come from human PBMCs harvested according to common protocols, the expectation is that the correlation coefficients reveal a high degree of similarity in general (i.e., the expression levels of the majority of the transcript sequences are similar in all samples analyzed). To summarize the similarity of samples, the average of the r2-values between all MAS signals of each sample and the other samples in the study was calculated and plotted in a heat map to facilitate rapid visualization. The closer the value of average r2 is to 1, the more alike the sample is to the other samples within the analysis. Low average r2-values indicate that the gene expression profile of the sample is an “outlier” in terms of overall gene expression patterns. Outlier status can indicate either that the sample has a gene expression profile that deviates significantly from the other samples within the analysis, or that the technical quality of the sample was of inferior quality.

### Example 5

### Clinical Study Protocol Summary

PBMCs were isolated from peripheral blood of 20 disease-free volunteers (12 females and 8 males) and 45 renal cell carcinoma patients (18 females and 27 males) participating in the phase II study. Consent for the pharmacogenomic portion of the clinical study was received and the project was approved by the local Institutional Review Boards at the participating clinical sites. The RCC tumors were classified at each site as conventional (clear cell) carcinomas (24), granular (1), papillary (3), or mixed subtypes (7). Classifications for ten tumors were not identified. The 45 patients who signed informed consent for pharmacogenomic analysis of baseline PBMC expression profiles were also scored by the multivariate assessment method of Motzer. Of the consented patients enrolled in this study, 6 were assigned a favorable risk assessment, 17 patients possessed an intermediate risk score, and 22 patients received a poor prognosis classification in this study.

Patients with advanced cases of RCC were treated with one of 3 doses of CCI-779 (25 mg, 75 mg, 250 mg) administered as a 30 minute IV infusion once weekly for the duration of the trial. Clinical staging and size of residual, recurrent or metastatic disease were recorded prior to treatment and every 8 weeks following initiation of CCI-779 therapy. Tumor size was measured in centimeters and reported as the product of the longest diameter and its perpendicular. Measurable disease was defined as any bidimensionally measurable lesion where both diameters >1.0 cm by CT-scan, X-ray or palpation. Tumor responses (complete response, partial response, minor response, stable disease or progressive disease) were determined by the sum of the products of the perpendicular diameters of all measurable lesions. The two main clinical outcome measures utilized in the present pharmacogenomic study were time to progression (TTP) and survival or time to death (TTD). TTP was defined as the interval from the date of initial CCI-779 treatment until the first day of measurement of progressive disease, or censored at the last date known as progression-free. Survival or TTD was defined as the interval from date of initial CCI-779 treatment to the time of death, or censored at the last date known alive.

### Example 6

### Statistical Analyses

Unsupervised hierarchical clustering of genes and/or arrays on the basis of similarity of their expression profiles was performed using the procedure of Eisen, et al., PROC NATL ACAD SCI U.S.A., 95:14863-14868 (1998). In these analyses only those transcripts meeting a non-stringent data reduction filter were used (at least 1 present call, at least 1 frequency across the data set of greater than or equal to 10 ppm). Expression data were log transformed and standardized to have a mean value of zero and a variance of one, and hierarchical clustering results were generated using average linkage clustering with an uncentered correlation similarity metric.

To identify the disease-associated transcripts an average fold change and a Student's t test was used to compare normal PBMC expression profiles to renal carcinoma PBMC profiles.

With respect to correlation of clinical outcome to pretreatment profiles, for simple univariate assessments of the relationships between pretreatment expression levels and continuous measures of clinical outcome, correlations between expression and TTP, and between expression and TTD, were calculated for each transcript using Spearman's rank correlations. Gene expression data were also assessed with censored measures of clinical outcomes (TTP, TTD) using a Cox proportional hazards regression model.

Survival data of various groups of patients were assessed by Kaplan Meier analysis, and significance was established using a Wilcoxon test.

Gene selection and supervised class prediction were performed using Genecluster version 2.0 which is described in Golub, et al., SCIENCE, 286: 531-537 (1999) and available from www.genome.wi.mit.edu/cancer/software/genecluster2.html. Those transcripts meeting a more stringent data reduction filter (at least 25% present calls, and an average frequency across all RCC PBMCs ≧5 ppm) were used to predict clinical outcome. This more stringent filter can avoid or minimize the inclusion of low level transcripts in the predictive models.

For nearest neighbor analysis all expression data in training sets and test sets were log transformed prior to analysis. In training sets of data, models containing increasing numbers of features (transcript sequences) were built using a two-sided approach (equal numbers of features in each class) with a S2N similarity metric that used median values for the class estimate. All comparisons were binary distinctions, and each model (with increasing numbers of features) was evaluated by leave one out cross validation, 10-fold cross validation, or 4-fold cross validation. Prediction of class membership in the test sets was performed using a k nearest neighbor algorithm which can also be found in Genecluster. See also Armstrong, et al., NATURE GENETICS, 30:41-47 (2002). For many predictions, the number of neighbors was set to k=3, the cosine distance measure used, and all k neighbors were given equal weights.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

What is claimed is:

1. A method for prognosis of renal cell carcinoma (RCC), said method comprising comparing an expression profile of one or more genes in a peripheral blood sample of an RCC patient of interest to at least one reference expression profile of said one or more genes,
wherein said one or more genes comprise a gene selected from Table 2 or 3, and
wherein the difference or similarity between said expression profile of the patient of interest and said at least one reference expression profile is indicative of prognosis of RCC in the patient of interest.

2. The method according to claim 1, wherein said gene selected from Table 2 or 3 is not PRKCD, MD-2, or VNN2.

3. The method according to claim 2, wherein the peripheral blood sample of the patient of interest is a whole blood sample or comprises enriched PBMCs.

4. The method according to claim 3, wherein said at least one reference expression profile comprises:
an average baseline peripheral blood expression profile of said one or more genes in RCC patients who have a first clinical outcome in response to an anti-cancer therapy, or
a plurality of profiles, each of which represents a baseline peripheral blood expression profile of said one or more genes in a different respective RCC patient who has the first or a second clinical outcome in response to the anti-cancer therapy.

5. The method according to claim 4, wherein the expression profile of the patient of interest is a baseline expression profile for the anti-tumor therapy.

6. The method according to claim 5, wherein the anti-tumor therapy is a CCI-779 therapy.

7. The method according to claim 6, wherein said one or more genes comprise a gene selected from Gene Nos. 1-7 of Table 2 and another gene selected from Gene Nos. 8-14 of Table 2, and the first and second outcomes are measured by patient TTD in response to the CCI-779 therapy.

8. The method according to claim 6, wherein said one or more genes comprise a gene selected from Gene Nos. 1-14 of Table 3 and another gene selected from Gene Nos. 15-28 of Table 3, and the first and second outcomes are measured by patient TTP in response to the CCI-779 therapy.

9. The method according to claim 6, wherein said one or more genes comprise a classifier selected from Table 4, and the expression profile of the patient of interest is compared to said at least one reference expression profile by using a k-nearest-neighbors or weighted voting algorithm.

10. The method according to claim 6, comprising the step of:
predicting if the patient of interest has the first or the second clinical outcome in response to the CCI-779 therapy.

11. A method of selecting a treatment for renal cell carcinoma (RCC), comprising the steps of:
providing prognoses of an RCC patient of interest for a plurality of treatments according to the method of claim 1; and
selecting a treatment from said plurality of treatments that has a favorable prognosis for the RCC patient of interest.

12. A system comprising:
a first storage medium including data that represent an expression profile of one or more genes in a peripheral blood sample of a patient who has a solid tumor;
a second storage medium including data that represent at least one reference expression profile of said one or more genes;
a program capable of comparing the expression profile to said at least one reference expression profile; and
a processor capable of executing the program, wherein said one or more genes comprise a gene selected from Tables 2 or 3, and said gene is not PRKCD, MD-2, or VNN2.

13. A kit for prognosis or selection of treatment of renal cell carcinoma (RCC), said kit comprising a probe for a gene selected from Table 2 or 3, wherein said gene is not PRKCD, MD-2, or VNN2.

14. A method for prognosis of solid tumors, said method comprising comparing an expression profile of one or more genes in a peripheral blood sample of a patient of interest to at least one reference expression profile of said one or more genes,
wherein the patient of interest has a solid tumor, and each of said one or more genes is differentially expressed in peripheral blood mononuclear cells (PBMCs) of a first class of patients relative to PBMCs of a second class of patients,
wherein both the first and second classes of patients have the solid tumor, and the first class of patients has a first clinical outcome and the second class of patients has a second clinical outcome,
wherein said one or more genes comprise a gene whose HG-U133A-determined PBMC expression profile in the first class and the second class of patients is correlated with a class distinction under a class-based correlation metric, said class distinction representing an idealized expression pattern of said gene in PBMCs of the first and second classes of patients, and
wherein the difference or similarity between said express profile of the patient of interest and said at least one reference expression profile is indicative of prognosis of the solid tumor in the patient of interest.

15. The method according to claim 14, wherein the first and the second clinical outcomes are outcomes to an anti-tumor therapy.

16. The method according to claim 15, wherein said HG-U133A-determined PBMC expression profile is a baseline expression profile for the anti-tumor therapy.

17. The method according to claim 16, wherein the solid tumor is renal cell carcinoma (RCC), and the peripheral blood sample of the patient of interest is a whole blood sample or comprises enriched PBMCs, and wherein said at least one reference expression profile comprises:
an average baseline peripheral blood expression profile of said one or more genes in patients who have the solid tumor and the first clinical outcome; or
a plurality of profiles, each of which represents a baseline peripheral blood expression profile of said one or more genes in a different respective patient who has the solid tumor and a clinical outcome selected from the group consisting of the first clinical outcome and the second clinical outcome.

18. The method according to claim 17, wherein the first and the second clinical outcomes are measured by TTD or TTP in response to a CCI-779 therapy.

19. The method according to claim 18, wherein said one or more genes comprise:
a gene selected from Gene Nos. 1-7 of Table 2 and another gene selected from Gene Nos. 8-14 of Table 2; or
a gene selected from Gene Nos. 1-14 of Table 3 and another gene selected from Gene Nos. 15-28 of Table 3.

20. The method according to claim 18, wherein said one or more genes comprise a classifier selected from Table 4, and said expression profile of the patient of interest is compared to said at least one reference expression profile by using a k-nearest-neighbors or weighted voting algorithm.
